(19) United States
(12) Patent Application Publication
(10) Pub. No.: US 2003/0194726 A1
(43) Pub. Date: Oct. 16, 2003

(54) Thermus oshimai nucleic acid polymerases

(75) Inventors: Elena V. Bolchakova, Union City, CA (US); James E. Rozzelle, San Francisco, CA (US)

Correspondence Address: MILA KASAN, PATENT DEPT., APPLIED BIOSYSTEMS, 850 LINCOLN CENTRE DRIVE, FOSTER CITY, CA, 94404, US

(73) Assignee: APPLERA CORPORATION, Foster City, CA

(21) Appl. No.: 10/303,109

(22) Filed: Nov. 22, 2002

Related U.S. Application Data
(60) Provisional application No. 60/334,798, filed on Nov. 30, 2001.

(51) Int. Cl. C12Q 1/68; C07H 21/04; C12P 19/34; C12N 9/22; C12P 21/02; C12N 5/06

(52) U.S. Cl. 435/6; 435/69.1; 435/91.2; 435/199; 435/320.1; 435/325; 536/23.2

(57) ABSTRACT

The invention provides nucleic acids and polypeptides for a nucleic acid polymerase enzyme from a thermophilic organism, Thermus oshimai. The invention also provides methods for using these nucleic acids and polypeptides.

### CROSS REFERENCE TO RELATED APPLICATIONS

This application claims a priority benefit under 35 U.S.C. § 119(e) from U.S. Patent Application No. 60/334,798, filed Nov. 30, 2001, which is incorporated herein by reference.

### FIELD OF THE INVENTION

The invention relates to nucleic acids and polypeptides for nucleic acid polymerase enzymes from the thermophilic bacterium Thermus oshimai.

### BACKGROUND OF THE INVENTION

DNA polymerases are naturally-occurring intracellular enzymes used by a cell for replicating DNA by reading one nucleic acid strand and manufacturing its complement. Enzymes having DNA polymerase activity catalyze the formation of a bond between the 3′ hydroxyl group at the growing end of a nucleic acid primer and the 5′ phosphate group of a newly added nucleotide triphosphate. Nucleotide triphosphates used for DNA synthesis are usually deoxyadenosine triphosphate (A), deoxythymidine triphosphate (T), deoxycytosine triphosphate (C) and deoxyguanosine triphosphate (G), but modified or altered versions of these nucleotides can also be used. The order in which the nucleotides are added is dictated by hydrogen-bond formation between A and T nucleotide bases and between G and C nucleotide bases.

Bacterial cells contain three types of DNA polymerases, termed polymerase I, II and III. DNA polymerase I is the most abundant polymerase and is generally responsible for certain types of DNA repair, including a repair-like reaction that permits the joining of Okazaki fragments during DNA replication. DNA Polymerase I is essential for the repair of DNA damage induced by UV irradiation and radiomimetic drugs. DNA Polymerase II is thought to play a role in repairing DNA damage that induces the SOS response. In mutants that lack both DNA Polymerase I and III, DNA Polymerase II repairs UV-induced lesions. DNA Polymerase I and II are monomeric polymerases while DNA Polymerase III is a multisubunit complex.

Enzymes having DNA polymerase activity are often used in vitro for a variety of biochemical applications including cDNA synthesis and DNA sequencing reactions. See Sambrook e al., Molecular Cloning: A Laboratory Manual (3rd ed. Cold Spring Harbor Laboratory Press, 2001, hereby incorporated by reference. DNA polymerases are also used for amplification of nucleic acids by methods such as the polymerase chain reaction (PCR) (Mullis et al., U.S. Pat. Nos. 4,683,195, 4,683,202, and 4,800,159, incorporated by reference) and RNA transcription-mediated amplification methods (e.g., Kacian et al., PCT Publication No. WO91/01384, incorporated by reference).

DNA amplification utilizes cycles of primer extension through the use of a DNA polymerase activity, followed by thermal denaturation of the resulting double-stranded nucleic acid in order to provide a new template for another round of primer annealing and extension. Because the high temperatures necessary for strand denaturation result in the irreversible inactivations of many DNA polymerases, the discovery and use of DNA polymerases able to remain active at temperatures above about 37° C. provides an advantage in cost and labor efficiency.

Thermostable DNA polymerases have been discovered in a number of thermophilic organisms including Thermus aquaticus, Thermus thermophilus, and species within the genera the Bacillus, Thermococcus, Sulfobus, and Pyrococcus. A full length thermostable DNA polymerase derived from Thermus aquaticus (Taq) has been described by Lawyer, et al., J. Biol. Chem. 264:6427-6437 (1989) and Gelfand et al, U.S. Pat. No. 5,466,591. The cloning and expression of truncated versions of that DNA polymerase are further described in Lawyer et al., in PCR Methods and Applications, 2:275-287 (1993), and Barnes, PCT Publication No. WO92/06188 (1992). Sullivan reports the cloning of a mutated version of the Taq DNA polymerase in EPO Publication No. 0482714A1 (1992). A DNA polymerase from Thermus thermophilus has also been cloned and expressed. Asakura et al., J. Ferment. Bioeng. (Japan), 74:265-269 (1993). However, while thermally stable DNA polymerases have been isolated, RNA polymerases that are thermally stable are not available. Moreover, the properties of the available DNA polymerases vary. Accordingly, new polymerases are needed that can synthesize both RNA and DNA and that have improved sequence discrimination, better salt tolerance, varying degrees of thermostability, improved tolerance for labeled or dideoxy nucleotides and other valuable properties.

### SUMMARY OF THE INVENTION

The invention provides nucleic acids and polypeptides for nucleic acid polymerase enzymes isolated from a thermophilic organism, Thermus oshimai. The strain used to clone the nucleic acids of this invention has been designated strain CB1 (Williams, R. A. D., Smith, K. E., Welch, S. G., and Micallef, J. Thermus oshimai sp. nov., Isolated from Hot Springs in Portugal, Iceland, and the Azores, and Comment on the Concept of a Limited Geographical Distrubution of Thermus Species. International Journal of Systematic Bacteriology, (1996) 46: 403-408.)

In one embodiment, the invention provides an isolated nucleic acid comprising SEQ ID NO:1, SEQ ID NO:2, SEQ ID NO:3, or SEQ ID NO:4, and complementary nucleic acids. In another embodiment, the invention provides an isolated nucleic acid encoding an amino acid sequence with at least 93% identity to SEQ ID NO:5, SEQ ID NO:6, SEQ ID NO:7 or SEQ ID NO:8. The invention also provides vectors comprising these isolated nucleic acids, including expression vectors comprising a promoter operably linked to these isolated nucleic acids. Host cells comprising such isolated nucleic acids and vectors are also provided by the invention, particularly host cells capable of expressing a thermostable polypeptide encoded by the nucleic acid, where the polypeptide has DNA or RNA polymerase activity.

In another embodiment, the invention provides an isolated nucleic acid encoding a derivative nucleic acid polymerase comprising amino acid sequence SEQ ID NO:5 that has a mutation that decreases 5-3′ exonuclease activity. The decreased 5-3′ exonuclease activity of the derivative nucleic acid polymerase is relative to a nucleic acid polymerase comprising amino acid sequence SEQ ID NO:5.

In another embodiment, the invention provides an isolated nucleic acid encoding a derivative nucleic acid polymerase comprising amino acid sequence SEQ ID NO:5 having a mutation that reduces discrimination against dideoxynucleotide triphosphates. The reduced discrimination against dideoxynucleotide triphosphates is relative to a nucleic acid polymerase comprising amino acid sequence SEQ ID NO:5.

The invention also provides isolated polypeptides that can include amino acid sequence SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 The isolated polypeptides provided by the invention can have an amino acid sequence with at least 93% sequence identity to SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28, which can, for example, have a DNA or RNA polymerase activity between 50,000 U/mg protein and 500,000 U/mg protein.

In another embodiment, the invention provides a derivative nucleic acid polymerase comprising amino acid sequence SEQ ID NO:5 that has a mutation that decreases 5-3′ exonuclease activity. The decreased 5-3′ exonuclease activity of the derivative nucleic acid polymerase is relative to a nucleic acid polymerase comprising amino acid sequence SEQ ID NO:5.

In another embodiment, the invention provides a derivative nucleic acid polymerase comprising amino acid sequence SEQ ID NO:5 having a mutation that reduces discrimination against dideoxynucleotide triphosphates. The reduced discrimination against dideoxynucleotide triphosphates is relative to a nucleic acid polymerase comprising amino acid sequence SEQ ID NO:5.

The invention further provides a method of synthesizing a nucleic acid that includes contacting a polypeptide comprising SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 with a nucleic acid under conditions sufficient to permit polymerization of the nucleic acid. The nucleic acid is preferably DNA.

The invention further provides a method of synthesizing DNA from an RNA template that includes contacting a polypeptide comprising SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 with an RNA under conditions sufficient to permit polymerization of DNA from an RNA template.

The invention further provides a method for thermocyclic amplification of a nucleic acid that comprises contacting a nucleic acid with a thermostable polypeptide having SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 under conditions suitable for amplification of the nucleic acid, and amplifying the nucleic acid. Such amplification can involve, for example, by Reverse Transcription, Strand Displacement Amplification or Polymerase Chain Reaction.

The invention also provides a method of primer extending DNA comprising contacting a polypeptide comprising SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28 with a DNA under conditions sufficient to permit polymerization of DNA. Such primer extension can be performed, for example, to sequence DNA or to amplify DNA.

The invention further provides a method of making a nucleic acid polymerase comprising SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28. The method comprises incubating a host cell comprising a nucleic acid that encodes a polypeptide comprising S SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28, operably linked to a promoter under conditions sufficient for RNA transcription and translation. In one embodiment, the method uses a nucleic acid that comprises SEQ ID NO:1, SEQ ID NO:2, SEQ ID NO:3, or SEQ ID NO:4. The invention is also directed to a nucleic acid polymerase enzyme made by this method.

The invention also provides a kit that includes a container containing a nucleic acid polymerase enzyme of the invention. Such a nucleic acid polymerase enzyme can have an amino acid sequence comprising SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27 or SEQ ID NO:28. The kit can also contain an unlabeled nucleotide, a labeled nucleotide, a balanced mixture of nucleotides, a chain terminating nucleotide, a nucleotide analog, a buffer solution, a solution containing magnesium, a cloning vector, a restriction endonuclease, a sequencing primer, a solution containing reverse transcriptase, or a DNA or RNA amplification primer. Such kits can, for example, be adapted for performing DNA sequencing, DNA amplification, reverse transcription or primer extension reactions.

### DESCRIPTION OF THE FIGURE

FIG. 1 provides an amino acid alignment of DNA polymerase sequences from Thermus aquaticus (Taq), Thermus thermophilus (Tth), Thermus filiformis (Tfi), and Thermus oshimai strain CB1 (Tosh). There are 60 amino acid positions where the deduced amino acid sequence of Thermus oshimai nucleic acid polymerase enzyme is different from all the other DNA Polymerase I sequences. These positions are indicated in boldface. Thermus oshimai nucleic acid polymerase enzyme also has a different starting postion from the others in that it has three fewer amino acids at its N terminus, also indicated in boldface.

### DETAILED DESCRIPTION OF THE INVENTION

The present invention relates to nucleic acid and amino acid sequences encoding a nucleic acid polymerase enzyme from thermophilic organisms. In particular, the present invention provides nucleic acid polymerase enzyme from Thermus oshimai, for example, from Thermus oshimai, strain CB1. The Thermus oshimai polymerase polypeptides of the invention can be used in a variety of procedures, including DNA synthesis, DNA primer extension, reverse transcription, DNA sequencing and nucleic acid amplification procedures.

Definitions

The term “amino acid sequence” refers to the positional arrangement and identity of amino acids in a peptide, polypeptide or protein molecule. Use of the term “amino acid sequence” is not meant to limit the amino acid sequence to the complete, native amino acid sequence of a peptide, polypeptide or protein.

“Chimeric” is used to indicate that a DNA sequence, such as a vector or a gene, is comprised of more than one DNA sequence of distinct origin that is fused by recombinant DNA techniques to another DNA sequence, resulting in a longer DNA sequence that does not occur naturally.

The term “coding region” refers to the nucleic acid segment that codes for a protein of interest. The coding region of a protein is bounded on the 5′ side by the nucleotide triplet “ATG” that encodes the initiator methionine and on the 3′ side by one of the three triplets that specify stop codons (i.e., TAA, TAG, TGA).

“Constitutive expression” refers to expression using a constitutive promoter.

“Constitutive promoter” refers to a promoter that is able to express the gene that it controls in all, or nearly all, phases of the life cycle of the cell.

“Complementary” or “complementarity” is used to define the degree of base-pairing or hybridization between nucleic acids. For example, as is known to one of skill in the art, adenine (A) can form hydrogen bonds or base pair with thymine (T) and guanine (G) can form hydrogen bonds or base pair with cytosine (C). Hence, A is complementary to T while G is complementary to C. Complementarity may be complete when all bases in a double-stranded nucleic acid are base paired. Alternatively, complementarity may be “partial,” when only some of the bases in a nucleic acid are matched according to the base pairing rules. The degree of complementarity between nucleic acid strands has an effect on the efficiency and strength of hybridization between nucleic acid strands.

The “derivative” of a reference nucleic acid, protein, polypeptide or peptide, is a nucleic acid, protein, polypeptide or peptide, respectively, with a related but different sequence or chemical structure than the respective reference nucleic acid, protein, polypeptide or peptide. A derivative nucleic acid, protein, polypeptide or peptide is generally made purposefully to enhance or incorporate some chemical, physical or functional property that is absent or only weakly present in the reference nucleic acid, protein, polypeptide or peptide. A derivative nucleic acid generally can differ in nucleotide sequence from a reference nucleic acid whereas a derivative protein, polypeptide or peptide can differ in amino acid sequence from the reference protein, polypeptide or peptide, respectively. Such sequence differences can be one or more substitutions, insertions, additions, deletions, fusions and truncations, which can be present in any combination. Differences can be minor (e.g., a difference of one nucleotide or amino acid) or more substantial. However, the sequence of the derivative is not so different from the reference that one of skill in the art would not recognize that the derivative and reference are related in structure and/or function. Generally, differences are limited so that the reference and the derivative are closely similar overall and, in many regions, identical. A “variant” differs from a “derivative” nucleic acid, protein, polypeptide or peptide in that the variant can have silent structural differences that do not significantly change the chemical, physical or functional properties of the reference nucleic acid, protein, polypeptide or peptide. In contrast, the differences between the reference and derivative nucleic acid, protein, polypeptide or peptide are intentional changes made to improve one or more chemical, physical or functional properties of the reference nucleic acid, protein, polypeptide or peptide.

The terms “DNA polymerase activity,” “synthetic activity” and “polymerase activity” are used interchangeably and refer to the ability of an enzyme to synthesize new DNA strands by the incorporation of deoxynucleoside triphosphates. A protein that can direct the synthesis of new DNA strands by the incorporation of deoxynucleoside triphosphates in a template-dependent manner is said to be “capable of DNA synthetic activity.”

The term “5 exonuclease activity” refers to the presence of an activity in a protein that is capable of removing nucleotides from the 5′ end of a nucleic acid.

The term “3′ exonuclease activity” refers to the presence of an activity in a protein that is capable of removing nucleotides from the 3′ end of a nucleic acid.

“Expression” refers to the transcription and/or translation of an endogenous or exogeneous gene in an organism. Expression generally refers to the transcription and stable accumulation of mRNA. Expression may also refer to the production of protein.

“Expression cassette” means a nucleic acid sequence capable of directing expression of a particular nucleotide sequence. Expression cassettes generally comprise a promoter operably linked to the nucleotide sequence to be expressed (e.g., a coding region) that is operably linked to termination signals. Expression cassettes also typically comprise sequences required for proper translation of the nucleotide sequence. The expression cassette comprising the nucleotide sequence of interest may be chimeric, meaning that at least one of its components is heterologous with respect to at least one of its other components. The expression of the nucleotide sequence in the expression cassette may be under the control of a constitutive promoter or under control of an inducible promoter that initiates transcription only when the host cell is exposed to some particular external stimulus. In the case of a multicellular organism, the promoter can also be specific to a particular tissue or organ or stage of development.

The term “gene” is used broadly to refer to any segment of nucleic acid associated with a biological function. The term “gene” encompasses the coding region of a protein, polypeptide, peptide or structural RNA. The term “gene” also includes sequences up to a distance of about 2 kb on either end of a coding region. These sequences are referred to as “flanking” sequences or regions (these flanking sequences are located 5′ or 3′ to the non-translated sequences present on the mRNA transcript). The 5′ flanking region may contain regulatory sequences such as promoters and enhancers or other recognition or binding sequences for proteins that control or influence the transcription of the gene. The 3′ flanking region may contain sequences that direct the termination of transcription, post-transcriptional cleavage and polyadenylation as well as recognition sequences for other proteins. A protein or polypeptide encoded in a gene can be full length or any portion thereof, so that all activities or functional properties are retained, or so that only selected activities (e.g., enzymatic activity, ligand binding, or signal transduction) of the full-length protein or polypeptide are retained. The protein or polypeptide can include any sequences necessary for the production of a proprotein or precursor polypeptide. The term “native gene” refers to gene that is naturally present in the genome of an untransformed cell.

“Genome” refers to the complete genetic material that is naturally present in an organism and is transmitted from one generation to the next.

The terms “heterologous nucleic acid,” or “exogenous nucleic acid” refer to a nucleic acid that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring nucleic acid. Thus, the terms refer to a nucleic acid segment that is foreign or heterologous to the cell, or normally found within the cell but in a position within the cell or genome where it is not ordinarily found.

The term “homology” refers to a degree of similarity between a nucleic acid and a reference nucleic acid or between a polypeptide and a reference polypeptide. Homology may be partial or complete. Complete homology indicates that the nucleic acid or amino acid sequences are identical. A partially homologous nucleic acid or amino acid sequence is one that is not identical to the reference nucleic acid or amino acid sequence. Hence, a partially homologous nucleic acid has one or more nucleotide differences in its sequence relative to the nucleic acid to which it is being compared. The degree of homology can be determined by sequence comparison. Alternatively, as is well understood by those skilled in the art, DNA-DNA or DNA-RNA hybridization, under various hybridization conditions, can provide an estimate of the degree of homology between nucleic acids, (see, e.g., Haines and Higgins (eds.), Nucleic Acid Hybridization, IRL Press, Oxford, U.K.).

“Hybridization” refers to the process of annealing complementary nucleic acid strands by forming hydrogen bonds between nucleotide bases on the complementary nucleic acid strands. Hybridization, and the strength of the association between the nucleic acids, is impacted by such factors as the degree of complementary between the hybridizing nucleic acids, the stringency of the conditions involved, the Tₘ of the formed hybrid, and the G:C ratio within the nucleic acids.

“Inducible promoter” refers to a regulated promoter that can be turned on in one or more cell types by an external stimulus, such as a chemical, light, hormone, stress, temperature or a pathogen.

An “initiation site” is region surrounding the position of the first nucleotide that is part of the transcribed sequence, which is defined as position +1. All nucleotide positions of the gene are numbered by reference to the first nucleotide of the transcribed sequence, which resides within the initiation site. Downstream sequences (i.e., sequences in the 3′ direction) are denominated positive, while upstream sequences (i.e., sequences in the 5′ direction) are denominated negative.

An “isolated” or “purified” nucleic acid or an “isolated” or “purified” polypeptide is a nucleic acid or polypeptide that, by the hand of man, exists apart from its native environment and is therefore not a product of nature. An isolated nucleic acid or polypeptide may exist in a purified form or may exist in a non-native environment such as, for example, within a transgenic host cell.

The term “invader oligonucleotide” refers to an oligonucleotide that contains sequences at its 3′ end that are substantially the same as sequences located at the 5′ end of a probe oligonucleotide. These regions will compete for hybridization to the same segment along a complementary target nucleic acid.

The term “label” refers to any atom or molecule that can be used to provide a detectable (preferably quantifiable) signal, and that can be attached to a nucleic acid or protein. Labels may provide signals detectable by fluorescence, radioactivity, colorimetry, gravimetry, X-ray diffraction or absorption, magnetism, enzymatic activity, and the like.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the reference sequence explicitly indicated.

The term “oligonucleotide,” as used herein, is defined as a molecule comprised of two or more deoxyribonucleotides or ribonucleotides, preferably more than three, and usually more than ten or fifteen. There is no precise upper limit on the size of an oligonucleotide. However, in general, an oligonucleotide is shorter than about 250 nucleotides, preferably shorter than about 200 nucleotides and more preferably shorter than about 100 nucleotides. The exact size will depend on many factors, which in turn depends on the ultimate function or use of the oligonucleotide. The oligonucleotide may be generated in any manner, including chemical synthesis, DNA replication, reverse transcription, or a combination thereof.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably linked” means joined as part of the same nucleic acid molecule, so that the function of one is affected by the other. In general, “operably linked” also means that two or more nucleic acids are suitably positioned and oriented so that they can function together. Nucleic acids are often operably linked to permit transcription of a coding region to be initiated from the promoter. For example, a regulatory sequence is said to be “operably linked to” or “associated with” a DNA sequence that codes for an RNA or a polypeptide if the two sequences are situated such that the regulatory sequence affects expression of the coding region (i.e., that the coding sequence or functional RNA is under the transcriptional control of the promoter). Coding regions can be operably-linked to regulatory sequences in sense or antisense orientation.

The term “probe oligonucleotide” refers to an oligonucleotide that interacts with a target nucleic acid to form a cleavage structure in the presence or absence of an invader oligonucleotide. When annealed to the target nucleic acid, the probe oligonucleotide and target form a cleavage structure and cleavage occurs within the probe oligonucleotide. The presence of an invader oligonucleotide upstream of the probe oligonucleotide can shift the site of cleavage within the probe oligonucleotide (relative to the site of cleavage in the absence of the invader).

“Promoter” refers to a nucleotide sequence, usually upstream (5′) to a coding region, which controls the expression of the coding region by providing the recognition site for RNA polymerase and other factors required for proper transcription. “Promoter” includes but is not limited a minimal promoter that is a short DNA segment comprised of a TATA-box. Hence, a promoter includes other sequences that serve to specify the site of transcription initiation and control or regulate expression, for example, enhancers. Accordingly, an “enhancer” is a DNA segment that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Promoters may be derived in their entirety from a native gene, or be composed of different elements derived from different promoters found in nature, or even be comprised of synthetic DNA segments. A promoter may also contain DNA segments that are involved in the binding of protein factors that control the effectiveness of transcription initiation in response to physiological or developmental conditions.

The terms “protein,” “peptide” and “polypeptide” are used interchangeably herein.

“Regulatory sequences” and “regulatory elements” refer to nucleotide sequences that control some aspect of the expression of nucleic acid sequences. Such sequences or elements can be located upstream (5′ non-coding sequences), within, or downstream (3′ non-coding sequences) of a coding sequence. “Regulatory sequences” and “regulatory elements” influence the transcription, RNA processing or stability, or translation of the associated coding sequence. Regulatory sequences include enhancers, introns, promoters, polyadenylation signal sequences, splicing signals, termination signals, and translation leader sequences. Regulatory sequences also include natural and synthetic sequences.

As used herein, the term “selectable marker” refers to a gene that encodes an observable or selectable trait that is expressed and can be detected in an organism having that gene. Selectable markers are often linked to a nucleic acid of interest that may not encode an observable trait in order to trace or select for the presence of the nucleic acid of interest. Any selectable marker known to one of skill in the art can be used with the nucleic acids of the invention. Some selectable markers allow the host to survive under circumstances where, without the marker, the host would otherwise die. Examples of selectable markers include antibiotic resistance, for example, tetracycline or ampicillin resistance.

As used herein the term “stringency” is used to define the conditions of temperature, ionic strength, and the presence of other compounds such as organic solvents, under which nucleic acid hybridizations are conducted. With “high stringency” conditions, nucleic acid base pairing will occur only between nucleic acids that have a high frequency of complementary base sequences. With “weak” or “low” stringency conditions nucleic acids the frequency of complementary sequences is usually less, so that nucleic acids with differing sequences can be detected and/or isolated.

The terms “substantially similar” and “substantially homologous” refer to nucleotide and amino acid sequences that represent functional equivalents of the instant inventive sequences. For example, altered nucleotide sequences that simply reflect the degeneracy of the genetic code but nonetheless encode amino acid sequences that are identical to the inventive amino acid sequences are substantially similar to the inventive sequences. In addition, amino acid sequences that are substantially similar to the instant sequences are those wherein overall amino acid identity is sufficient to provide an active, thermally stable nucleic acid polymerase. For example, amino acid sequences that are substantially similar to the sequences of the invention are those wherein the overall amino acid identity is 80% or greater, preferably 90% or greater, such as 91%, 92%, 93%, or 94%, and more preferably 95% or greater, such as 96%, 97%, 98%, or 99%, relative to the amino acid sequences of the invention.

A “terminating agent,” “terminating nucleotide” or “terminator” in relation to DNA synthesis or sequencing refers to compounds capable of specifically terminating a DNA sequencing reaction at a specific base, such compounds include but are not limited to, dideoxynucleosides having a 2′, 3′ dideoxy structure (e.g., ddATP, ddCTP, ddGTP and ddTTP).

“Thermostable” means that a nucleic acid polymerase remains active at a temperature greater than about 37° C. Preferably, the nucleic acid polymerases of the invention remain active at a temperature greater than about 42° C. More preferably, the nucleic acid polymerases of the invention remain active at a temperature greater than about 50° C. Even more preferably, the nucleic acid polymerases of the invention remain active after exposure to a temperature greater than about 60° C. Most preferably, the nucleic acid polymerases of the invention remain active despite exposure to a temperature greater than about 70° C.

A “transgene” refers to a gene that has been introduced into the genome by transformation and is stably maintained. Transgenes may include, for example, genes that are either heterologous or homologous to the genes of a particular organism to be transformed. Additionally, transgenes may comprise native genes inserted into a non-native organism, or chimeric genes. The term “endogenous gene” refers to a native gene in its natural location in the genome of an organism. A “foreign” or “exogenous” gene refers to a gene not normally found in the host organism that is introduced by gene transfer.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.” Transformation may be accomplished by a variety of means known to the art including calcium DNA co-precipitation, electroporation, viral infection, and the like.

The “variant” of a reference nucleic acid, protein, polypeptide or peptide, is a nucleic acid, protein, polypeptide or peptide, respectively, with a related but different sequence than the respective reference nucleic acid, protein, polypeptide or peptide. The differences between variant and reference nucleic acids, proteins, polypeptides or peptides are silent or conservative differences. A variant nucleic acid differs from a reference nucleic acid in nucleotide sequence whereas a variant nucleic acid, protein, polypeptide or peptide differs in amino acid sequence from the reference protein, polypeptide or peptide, respectively. A variant and reference nucleic acid, protein, polypeptide or peptide may differ in sequence by one or more substitutions, insertions, additions, deletions, fusions and truncations, which may be present in any combination. Differences can be minor (e.g., a difference of one nucleotide or amino acid) or more substantial. However, the structure and function of the variant is not so different from the reference that one of skill in the art would not recognize that the variant and reference are related in structure and/or function. Generally, differences are limited so that the reference and the variant are closely similar overall and, in many regions, identical.

The term “vector” is used to refer to a nucleic acid that can transfer another nucleic acid segment(s) into a cell. A “vector” includes, inter alia, any plasmid, cosmid, phage or nucleic acid in double- or single-stranded, linear or circular form that may or may not be self-transmissible or mobilizable. It can transform prokaryotic or eukaryotic host cells either by integration into the cellular genome or by existing extrachromosomally (e.g., autonomous replicating plasmid with an origin of replication). Vectors used in bacterial systems often contain an origin of replication that allows the vector to replicate independently of the bacterial chromosome. The term “expression vector” refers to a vector containing an expression cassette.

The term “wild-type” refers to a gene or gene product that has the characteristics of that gene or gene product when isolated from a naturally occurring source. A wild-type gene is the gene form most frequently observed in a population and thus arbitrarily is designated the “normal” or “wild-type” form of the gene. In contrast, the term “variant” or “derivative” refers to a gene or gene product that displays modifications in sequence and or functional properties (i.e., altered characteristics) when compared to the wild-type gene or gene product. Naturally occurring derivatives can be isolated. They are identified by the fact that they have altered characteristics when compared to the wild-type gene or gene product.

Nucleic Acids Encoding the Polymerases of the Invention

The invention provides isolated nucleic acids encoding Thermus oshimai nucleic acid polymerases, as well as derivative, fragment and variant nucleic acids thereof that encode active, thermally stable nucleic acid polymerases. Thus, one aspect of the invention relates to the nucleic acid polymerases from Thermus oshimai, strain CB1. Any nucleic acid encoding amino acid sequence SEQ ID NO:5, SEQ ID NO:6, SEQ ID NO:7 and SEQ ID NO:8, which are amino acid sequences for Thermus oshimai wild type and several derivative polymerases, is also contemplated by the present invention.

In one embodiment, the invention provides a nucleic acid of SEQ ID NO:1, a wild type Thermus oshimai nucleic acid encoding a nucleic acid polymerase enzyme:

| | |
|---|---|
| 1 | ATGCTGCCCC TCTTTGAGCC CAAGGGCCGG GTCCTCCTGG TGGACGGCCA |
| 51 | CCACCTGGCC TACCGCACCT TTTTCGCCCT CAAGGGCCTC ACCACCAGCC |
| 101 | GGGGCGAGCC CGTGCAGGCG GTTTATGGCT TCGCCAAAAG CCTCCTCAAG |
| 151 | GCCCTGAAGG AGGATGGGGA GGTGGCCATC GTGGTCTTTG ACGCCAAGGC |
| 201 | CCCCTCCTTC CGCCACGAGG CCTACGAGGC CTACAAGGCG GGCCGGGCCC |
| 251 | CCACCCCGGA GGACTTTCCC CGGCAGCTCG CCCTCATCAA GGAGCTGGTG |
| 301 | GACCTTTTGG GCCTCGTGCG CCTTGAGGTC CCGGGCTTTG AGGCGGACGA |
| 351 | TGTCCTCGCC ACCCTGGCCA AGAAGGCAGA AAGGGAGGGG TACGAGGTGC |
| 401 | GCATCCTGAG CGCGGACCGC GACCTCTACC AGCTCCTTTC CGACCGGATC |
| 451 | CACCTCCTCC ACCCCGAGGG GGAGGTCCTG ACCCCCGGGT GGCTCCAGGA |
| 501 | GCGCTACGGC CTCTCCCCGG AGAGGTGGGT GGAGTACCGG GCCCTGGTGG |
| 551 | GGGACCCTTC GGACAACCTC CCCGGGGTGC CCGGCATCGG GGAGAAGACC |
| 601 | GCCCTGAAGC TCCTGAAGGA GTGGGGTAGC CTGGAAGCGA TTCTAAAGAA |
| 651 | CCTGGACCAG GTGAAGCCGG AAAGGGTGCG GGAGGCCATC CGGAATAACC |
| 701 | TGGATAAGCT CCAGATGTCC CTGGAGCTTT CCCGCCTCCG CACCGACCTC |
| 751 | CCCCTGGAGG TGGACTTCGC CAAGAGGCGG GAGCCCGACT GGGAGGGGCT |
| 801 | TAAGGCCTTT TTGGAGCGGC TTGAGTTCGG AAGCCTCCTC CACGAGTTCG |
| 851 | GCCTTCTGGA GGCCCCCAAG GAGGCGGAGG AGGCCCCCTG GCCCCCGCCT |
| 901 | GGAGGGGCCT TTTTGGGCTT CCTCCTCTCC CGCCCCGAGC CCATGTGGGC |
| 951 | GGAGCTTTTG GCCCTGGCGG GGGCCAAGGA GGGGCGGGTC CATCGGGCGG |
| 1001 | AAGACCCCGT GGGGGCCCTA AAGGACCTGA AGGAGATCCG GGGCCTCCTC |
| 1051 | GCCAAGGACC TCTCGGTCCT GGCCCTGAGG GAGGGCCGGG AGATCCCGCC |
| 1101 | GGGGGACGAC CCCATGCTCC TCGdCTACCT CCTGGACCCG GGGAACACCA |
| 1151 | ACCCCGAGGG GGTGGCCCGG CGGTACGGGG GGGAGTGGAA GGAGGACGCC |
| 1201 | GCCGCCCGGG CCCTCCTTTC GGAAAGGCTC TGGCAGGCCC TTTACCCCCG |
| 1251 | GGTGGCGGAG GAGGAAAGGC TCCTTTGGCT CTACCGGGAG GTGGAGCGGC |
| 1301 | CCCTCGCCCA GGTCCTCGCC CACATGGAGG CCACGGGGGT GCGGCTGGAT |
| 1351 | GTGCCCTACC TGGAGGCCCT TTCCCAGGAG GTGGCCTTTG AGCTGGAGCG |
| 1401 | CCTCGAGGCC GAGGTCCACC GCCTGGCGGG CCACCCCTTC AACCTGAACT |
| 1451 | CTAGGGACCA GCTGGAGCGG GTCCTCTTTG ACGAGCTCGG CCTACCCCCC |
| 1501 | ATCGGCAAGA CGGAGAAGAC GGGCAAGCGC TCCACCAGCG CCGCCGTCCT |
| 1551 | GGAGCTCTTA AGGGAGGCCC ACCCCATCGT GGGGCGGATC CTGGAGTACC |
| 1601 | GGGAGCTCAT GAAGCTCAAG AGCACCTACA TAGACCCCCT CCCCAGGCTG |
| 1651 | GTCCACCCCA AAACCGGCCG GCTCCACACC CGCTTCAACC AGACGGCCAC |
| 1701 | CGCCACGGGC CGCCTCTCCA GCTCCGACCC CAACCTGCAG AACATCCCCG |
| 1751 | TGCGCACCCC CTTAGGCCAG CGCATCCGCA AGGCCTTCAT TGCCGAGGAG |
| 1801 | GGCCATCTCC TGGTGGCCCT GGACTATAGC CAGATCGAGC TCCGGGTCCT |
| 1851 | CGCCCACCTC TCGGGGGACG AGAACCTCAT CCGGGTCTTC CGGGAAGGGA |
| 1901 | AGGACATCCA CACCGAGACC GCCGCCTGGA TGTTCGGCGT GCCCCCCGAG |
| 1951 | GGGGTGGACG GGGCCATGCG CCGGGCGGCC AAGACGGTGA ACTTCGGGGT |
| 2001 | GCTCTACGGC ATGTCCGCCC ACCGCCTCTC CCAGGAGCTC TCCATCCCCT |
| 2051 | ACGAGGAGGC GGCGGCCTTC ATCGAGCGCT ACTTCCAGAG CTTCCCCAAG |
| 2101 | GTGCGGGCCT GGATCGCCAA AACCTTGGAG GAGGGGCGGA AGAAGGGGTA |
| 2151 | CGTGGAGACC CTCTTCGGCC GCCGCCGCTA CGTGCCCGAC CTCAACGCCC |
| 2201 | GGGTGAAGAG CGTGCGGGAG GCGGCGGAGC GCATGGCCTT CAACATGCCC |
| 2251 | GTGCAGGGCA CCGCCGCGGA CCTCATGAAG CTGGCCATGG TGAAGCTCTT |
| 2301 | CCCCAGGCTC AGGCCCTTGG GCGTTCGCAT CCTCCTCCAG GTGCACGACG |
| 2351 | AGCTGGTCTT GGAGGCCCCA AAGGCGCGGG CGGAGGAGGC CGCCCAGTTG |
| 2401 | GCCAAGGAGA CCATGGAAGG GGTTTACCCC CTCTCCGTCC CCCTGGAGGT |
| 2451 | GGAGGTGGGG ATGGGGGAGG ACTGGCTTTC CGCCAAGGCC TAG |

In another embodiment, the invention provides a nucleic acid of SEQ ID NO:2, a derivative Thermus oshimai nucleic acid having GAC (encoding Asp) at positions 127-129 (codon 43) in place of GGC (encoding Gly). SEQ ID NO:2 is provided below:

| | |
|---|---|
| 1 | ATGCTCCCCC TCTTTGAGCC CAAGGGCCGG GTCCTCCTGG TGGACGGCCA |
| 51 | CCACCTGGCC TACCGCACCT TTTTCGCCCT CAAGGGCCTC ACCACCAGCC |
| 101 | GGGGCGAGCC CGTGCAGGCG GTTTATGACT TCGCCAAAAG CCTCCTCAAG |
| 151 | GCCCTGAAGG AGGATGGGGA GGTGGCCATC GTGGTCTTTG ACGCCAAGGC |
| 201 | CCCCTCCTTC CGCCACGACG CCTACGAGGC CTACAAGGCG GGCCGGGCCC |
| 251 | CCACCCCGGA GGACTTTCCC CGGCAGCTCG CCCTCATCAA GGAGCTGGTG |
| 301 | GACCTTTTGG GCCTCGTGCG CCTTGAGGTC CCGGGCTTTG AGGCGGACGA |
| 351 | TGTCCTCGCC ACCCTGGCCA AGAAGGCAGA AAGGGAGGGG TACGAGGTGC |
| 401 | GCATCCTGAG CGCGGACCGC GACCTCTACC AGCTCCTTTC CGACCGGATC |
| 451 | CACCTCCTCC ACCCCGAGGG GGAGGTCCTG ACCCCCGGGT GGCTCCAGGA |
| 501 | GCGCTACGGC CTCTCCCCGG AGAGGTGGGT GGAGTACCGG GCCCTGGTGG |
| 551 | GGGACCCTTC GGACAACCTC CCCGGGGTGC CCGGCATCGG GGAGAAGACC |
| 601 | GCCCTGAAGC TCCTGAAGGA GTGGGGTAGC CTGGAAGCGA TTCTAAAGAA |
| 651 | CCTGGACCAG GTGAAGCCGG AAAGGGTGCG GGAGGCCATC CGGAATAACC |
| 701 | TGGATAAGCT CCAGATGTCC CTGGAGCTTT CCCGCCTCCG CACCGACCTC |
| 751 | CCCCTGGAGG TGGACTTCGC CAAGAGGCGG GAGCCCGACT GGGAGGGGCT |
| 801 | TAAGGCCTTT TTGGAGCGGC TTGAGTTCGG AAGCCTCCTC CACGAGTTCG |
| 851 | GCCTTCTGGA GGCCCCCAAG GAGGCGGAGG AGGCCCCCTG GCCCCCGCCT |
| 901 | GGAGGGGCCT TTTTGGGCTT CCTCCTCTCC CGCCCCGAGC CCATGTGGGC |
| 951 | GGAGCTTTTG GCCCTGGCGG GGGCCAAGGA GGGGCGGGTC CATCGGGCGG |
| 1001 | AAGACCCCGT GGGGGCCCTA AAGGACCTGA AGGAGATCCG GGGCCTCCTC |
| 1051 | GCCAAGGACC TCTCGGTCCT GGCCCTGAGG GAGGGCCGGG AGATCCCGCC |
| 1101 | GGGGGACGAC CCCATGCTCC TCGCCTACCT CCTGGACCCG GGGAACACCA |
| 1151 | ACCCCGAGGG GGTGGCCCGG CGGTACGGGG GGGAGTGGAA GGAGGACGCC |
| 1201 | GCCGCCCGGG CCCTCCTTTC GGAAAGGCTC TGGCAGGCCC TTTACCCCCG |
| 1251 | GGTGGCGGAG GAGGAAAGGC TCCTTTGGCT CTACCGGGAG GTGGAGCGGC |
| 1301 | CCCTCGCCCA GGTCCTCGCC CACATGGAGG CCACGGGGGT GCGGCTGGAT |
| 1351 | GTGCCCTACC TGGAGGCCCT TTCCCAGGAG GTGGCCTTTG AGCTGGAGCG |
| 1401 | CCTCGAGGCC GAGGTCCACC GCCTGGCGGG CCACCCCTTC AACCTGAACT |
| 1451 | CTAGGGACCA GCTGGAGCGG GTCCTCTTTG ACGAGCTCGG CCTACCCCCC |
| 1501 | ATCGGCAAGA CGGAGAAGAC GGGCAAGCGC TCCACCAGCG CCGCCGTCCT |
| 1551 | GGAGCTCTTA AGGGAGGCCC ACCCCATCGT GGGGCGGATC CTGGAGTACC |
| 1601 | GGGAGCTCAT GAAGCTCAAG AGCACCTACA TAGACCCCCT CCCCAGGCTG |
| 1651 | GTCCACCCCA AAACCGGCCG GCTCCACACC CGCTTCAACC AGACGGCCAC |
| 1701 | CGCCACGGGC CGCCTCTCCA GCTCCGACCC CAACCTGCAG AACATCCCCG |
| 1751 | TGCGCACCCC CTTAGGCCAG CGCATCCGCA AGGCCTTCAT TGCCGAGGAG |
| 1801 | GGCCATCTCC TGGTGGCCCT GGACTATAGC CAGATCGAGC TCCGGGTCCT |
| 1851 | CGCCCACCTC TCGGGGGACG AGAACCTCAT CCGGGTCTTC CGGGAAGGGA |
| 1901 | AGGACATCCA CACCGAGACC GCCGCCTGGA TGTTCGGCGT GCCCCCCGAG |
| 1951 | GGGGTGGACG GGGCCATGCG CCGGGCGGCC AAGACGGTGA ACTTCGGGGT |
| 2001 | GCTCTACGGG ATGTCCGCCC ACCGCCTCTC CCAGGAGCTC TCCATCCCCT |
| 2051 | ACGAGGAGGC GGCGGCCTTC ATCGAGCGCT ACTTCCAGAG CTTCCCCAAG |
| 2101 | GTGCGGGCCT GGATCGCCAA AACCTTGGAG GAGGGGCGGA AGAAGGGGTA |
| 2151 | CGTGGAGACC CTCTTCGGCC GCCGCCGCTA CGTGCCCGAC CTCAACGCCC |
| 2201 | GGGTGAAGAG CGTGCGGGAG GCGGCGGAGC GCATGGCCTT CAACATGCCC |
| 2251 | GTGCAGGGCA CCGCCGCGGA CCTCATGAAG CTGGCCATGG TGAAGCTCTT |
| 2301 | CCCCAGGCTC AGGCCCTTGG GCGTTCGCAT CCTCCTCCAG GTGCACGACG |
| 2351 | AGCTGGTCTT GGAGGCCCCA AAGGCGCGGG CGGAGGAGGC CGCCCAGTTG |
| 2401 | GCCAAGGAGA CCATGGAAGG GGTTTACCCC CTCTCCGTCC CCCTGGAGGT |
| 2451 | GGAGGTGGGG ATGGGGGAGG ACTGGCTTTC CGCCAAGGCC TAG |

In another embodiment, the invention provides a nucleic acid of SEQ ID NO:3, a derivative Thermus oshimai nucleic acid having TAC (encoding Tyr) in place of TTC (encoding Phe) at positions 1993-1995 (codon 665), indicated in boldface. SEQ ID NO:3 is provided below:

| | |
|---|---|
| 1 | ATGCTGCCCC TCTTTGAGCC CAAGGGCCGG GTCCTCCTGG TGGACGGCCA |
| 51 | CCACCTGGCC TACCGCACCT TTTTCGCCCT CAAGGGCCTC ACCACCAGCC |
| 101 | GGCGCGAGCC CGTGCAGGCG GTTTATGGCT TCGCCAAAAG CCTCCTCAAG |
| 151 | GCCCTGAAGG AGGATGGGGA GGTGGCCATC GTGGTCTTTG ACGCCAAGGC |
| 201 | CCCCTCCTTC CGCCACGAGG CCTACGAGGC CTACAAGGCG GGCCGGGCCC |
| 251 | CCACCCCGGA GGACTTTCCC CGGCAGCTCG CCCTCATCAA GGAGCTGGTG |
| 301 | GACCTTTTGG GCCTCGTGCG CCTTGAGGTC CCGGGCTTTG AGGCGGACGA |
| 351 | TGTCCTCGCC ACCCTGGCCA AGAAGGCAGA AAGGGAGGGG TACGAGGTGC |
| 401 | GCATCCTGAG CGCGGACCGC GACCTCTACC AGCTCCTTTC CGACCGGATC |
| 451 | CACCTCCTCC ACCCCGAGGG GGAGGTCCTG ACCCCCGGGT GGCTCCAGGA |
| 501 | GCGCTACGGC CTCTCCCCGG AGAGGTGGGT GGAGTACCGG GCCCTGGTGG |
| 551 | GGGACCCTTC GGACAACCTC CCCGGGGTGC CCGGCATCGG GGAGAAGACC |
| 601 | GCCCTGAAGC TCCTGAAGGA GTGGGGTAGC CTGGAAGCGA TTCTAAAGAA |
| 651 | CCTGGACCAG GTGAAGCCGG AAAGGGTGCG GGAGGCCATC CGGAATAACC |
| 701 | TGGATAAGCT CCAGATGTCC CTGGAGCTTT CCCGCCTCCG CACCGACCTC |
| 751 | CCCCTGGAGG TGGACTTCGC CAAGAGGCGG GAGCCCGACT GGGAGGGGCT |
| 801 | TAAGGCCTTT TTGGAGCGGC TTGAGTTCGG AAGCCTCCTC CACGAGTTCG |
| 851 | GCCTTCTGGA GGCCCCCAAG GAGGCGGAGG AGGCCCCCTG GCCCCCGCCT |
| 901 | GGAGGGGCCT TTTTGGGCTT CCTCCTCTCC CGCCCCGAGC CCATGTGGGC |
| 951 | GGAGCTTTTG GCCCTGGCGG GGGCCAAGGA GGGGCGGGTC CATCGGGCGG |
| 1001 | AAGACCCCGT GGGGGCCCTA AAGGACCTGA AGGAGATCCG GGGCCTCCTC |
| 1051 | GCCAAGGACC TCTCGGTCCT GGCCCTGAGG GAGGGCCGGG AGATCCCGCC |
| 1101 | GGGGGACGAC CCCATGCTCC TCGCCTACCT CCTGGACCCG GGGAACACCA |
| 1151 | ACCCCGAGGG GGTGGCCCGG CGGTACGGGG GGGAGTGGAA GGAGGACGCC |
| 1201 | GCCGCCCGGG CCCTCCTTTC GGAAAGGCTC TGGCAGGCCC TTTACCCCCG |
| 1251 | GGTGGCGGAG GAGGAAAGGC TCCTTTGGCT CTACCGGGAG GTGGAGCGGC |
| 1301 | CCCTCGCCCA GGTCCTCGCC CACATGGAGG CCACGGGGGT GCGGCTGGAT |
| 1351 | GTGCCCTACC TGGAGGCCCT TTCCCAGGAG GTGGCCTTTG AGCTGGAGCG |
| 1401 | CCTCGAGGCC GAGGTCCACC GCCTGGCGGG CCACCCCTTC AACCTGAACT |
| 1451 | CTAGGGACCA GCTGGAGCGG GTCCTCTTTG ACGAGCTCGG CCTACCCCCC |
| 1501 | ATCGGCAAGA CGGAGAAGAC GGGCAAGCGC TCCACCAGCG CCGCCGTCCT |
| 1551 | GGAGCTCTTA AGGGAGGCCC ACCCCATCGT GGGGCGGATC CTGGAGTACC |
| 1601 | GGGAGCTCAT GAAGCTCAAG AGCACCTACA TAGACCCCCT CCCCAGGCTG |
| 1651 | GTCCACCCCA AAACCGGCCG GCTCCACACC CGCTTCAACC AGACGGCCAC |
| 1701 | CGCCACGGGC CGCCTCTCCA GCTCCGACCC CAACCTGCAG AACATCCCCG |
| 1751 | TGCGCACCCC CTTAGGCCAG CGCATCCGCA AGGCCTTCAT TGCCGAGGAG |
| 1801 | GGCCATCTCC TGGTGGCCCT GGACTATAGC CAGATCGAGC TCCGGGTCCT |
| 1851 | CGCCCACCTC TCGGGGGACG AGAACCTCAT CCGGGTCTTC CGGGAAGGGA |
| 1901 | AGGACATCCA CACCGAGACC GCCGCCTGGA TGTTCGGCGT GCCCCCCGAG |
| 1951 | GGGGTGGACG GGGCCATGCG CCGGGCGGCC AAGACGGTGA ACTACGGGGT |
| 2001 | GCTCTACGGG ATGTCCGCCC ACCGCCTCTC CCAGGAGCTC TCCATCCCCT |
| 2051 | ACGAGGAGGC GGCGGCCTTC ATCGAGCGCT ACTTCCAGAG CTTCCCCAAG |
| 2101 | GTGCGGGCCT GGATCGCCAA AACCTTGGAG GAGGGGCGGA AGAAGGGGTA |
| 2151 | CGTGGAGACC CTCTTCGGCC GCCGCCGCTA CGTGCCCGAC CTCAACGCCC |
| 2201 | GGGTGAAGAG CGTGCGGGAG GCGGCCCAGC CCATGGCCTT CAACATGCCC |
| 2251 | GTGCAGGGCA CCGCCGCGGA CCTCATGAAG CTGGCCATGG TGAAGCTCTT |
| 2301 | CCCCAGGCTC AGGCCCTTGG GCGTTCGCAT CCTCCTCCAG GTGCACGACG |
| 2351 | AGCTGGTCTT GGAGGCCCCA AAGGCGCGGG CGGAGGAGGC CGCCCAGTTG |
| 2401 | GCCAAGGAGA CCATGGAAGG GGTTTACCCC CTCTCCGTCC CCCTGGAGGT |
| 2451 | GGAGGTGGGG ATGGGGGAGG ACTGGCTTTC CGCCAAGGCC TAG |

In yet another embodiment, the invention provides a nucleic acid of SEQ ID NO:4, a derivative Thermus oshimai nucleic acid having GAC (encoding Asp) at positions 127-129 (codon 43) in place of GGC (encoding Gly) and having TAC (encoding Tyr) in place of TTC (encoding Phe) at positions 1993-1995. SEQ ID NO:4 is provided below:

| | |
|---|---|
| 1 | ATGCTGCCCC TCTTTGAGCC CAAGGGCCGG GTCCTCCTGG TGGACGGCCA |
| 51 | CCACCTGGCC TACCGCACCT TTTTCGCCCT CAAGGGCCTC ACCACCAGCC |
| 101 | GGGGCGAGCC CGTGCAGGCG GTTTATGACT TCGCCAAAAG CCTCCTCAAG |
| 151 | GCCCTGAAGG AGGATGGGGA GGTGGCCATC GTGGTCTTTG ACGCCAAGGC |
| 201 | CCCCTCCTTC CGCCACGAGG CCTACGAGGC CTACAAGGCG GGCCGGGCCC |
| 251 | CCACCCCGGA GGACTTTCCC CGGCAGCTCG CCCTCATCAA GGAGCTGGTG |
| 301 | GACCTTTTGG GCCTCGTGCG CCTTGAGGTC CCGGGCTTTG AGGCGGACGA |
| 351 | TGTCCTCGCC ACCCTGGCCA AGAAGGCAGA AAGGGAGGGG TACGAGGTGC |
| 401 | GCATCCTGAG CGCGGACCGC GACCTCTACC AGCTCCTTTC CGACCGGATC |
| 451 | CACCTCCTCC ACCCCGAGGG GGAGGTCCTG ACCCCCGGGT GGCTCCAGGA |
| 501 | GCGCTACGGC CTCTCCCCGG AGAGGTGGGT GGAGTACCGG GCCCTGGTGG |
| 551 | GGGACCCTTC GGACAACCTC CCCGGGGTGC CCGGCATCGG GGAGAAGACC |
| 601 | GCCCTGAAGC TCCTGAAGGA GTGGGGTAGC CTGGAAGCGA TTCTAAAGAA |
| 651 | CCTGGACCAG GTGAAGCCGG AAAGGGTGCG GGAGGCCATC CGGAATAACC |
| 701 | TGGATAAGCT CCAGATGTCC CTGGAGCTTT CCCGCCTCCG CACCGACCTC |
| 751 | CCCCTGGAGG TGGACTTCGC CAAGAGGCGG GAGCCCGACT GGGAGGGGCT |
| 801 | TAAGGCCTTT TTGGAGCGGC TTGAGTTCGG AAGCCTCCTC CACGAGTTCG |
| 851 | GCCTTCTGGA GGCCCCCAAG GAGGCGGAGG AGGCCCCCTG GCCCCCGCCT |
| 901 | GGAGGGGCCT TTTTGGGCTT CCTCCTCTCC CGCCCCGAGC CCATGTGGGC |
| 951 | GGAGCTTTTG GCCCTGGCGG GGGCCAAGGA GGGGCGGGTC CATCGGGCGG |
| 1001 | AAGACCCCGT GGGGGCCCTA AAGGACCTGA AGGACATCCG GGGCCTCCTC |
| 1051 | GCCAAGGACC TCTCGGTCCT GGCCCTCAGG GAGGGCCGGG AGATCCCGCC |
| 1101 | GGGGGACGAC CCCATGCTCC TCGCCTACCT CCTGGACCCG GGGAACACCA |
| 1151 | ACCCCGAGGG GGTGGCCCGG CGGTACGGGG GGGAGTGGAA GGAGGACGCC |
| 1201 | GCCGCCCGGG CCCTCCTTTC GGAAAGGCTC TGGCAGGCCC TTTACCCCCG |
| 1251 | GGTGGCGGAG GAGGAAAGGC TCCTTTGGCT CTACCGGGAG GTGGAGCGGC |
| 1301 | CCCTCGCCCA GGTCCTCGCC CACATGGAGG CCACGGGGGT GCGGCTGGAT |
| 1351 | GTGCCCTACC TGGAGGCCCT TTCCCAGGAG GTGGCCTTTG AGCTGGAGCG |
| 1401 | CCTCGAGGCC GAGGTCCACC GCCTGGCGGG CCACCCCTTC AACCTGAACT |
| 1451 | CTAGGGACCA GCTGGAGCGG GTCCTCTTTG ACGAGCTCGG CCTACCCCCC |
| 1501 | ATCGGCAAGA CGGAGAAGAC GGGCAAGCGC TCCACCAGCG CCGCCGTCCT |
| 1551 | GGAGCTCTTA AGGGAGGCCC ACCCCATCGT GGGGCGGATC CTGGAGTACC |
| 1601 | GGGAGCTCAT GAAGCTCAAG AGCACCTACA TAGACCCCCT CCCCAGGCTG |
| 1651 | GTCCACCCCA AAACCGGCCG GCTCCACACC CGCTTCAACC AGACGGCCAC |
| 1701 | CGCCACGGGC CGCCTCTCCA GCTCCGACCC CAACCTGCAG AACATCCCCG |
| 1751 | TGCGCACCCC CTTAGGCCAG CGCATCCGCA AGGCCTTCAT TGCCGAGGAG |
| 1801 | GGCCATCTCC TGGTGGCCCT GGACTATAGC CAGATCGAGC TCCGGGTCCT |
| 1851 | CGCCCACCTC TCGGGGGACG AGAACCTCAT CCGGGTCTTC CGGGAAGGGA |
| 1901 | AGGACATCCA CACCGAGACC GCCGCCTGGA TGTTCGGCGT GCCCCCCGAG |
| 1951 | GGGGTGGACG GGGCCATGCG CCGGGCGGCC AAGACGGTGA ACTACGGGGT |
| 2001 | GCTCTACGGG ATGTCCGCCC ACCGCCTCTC CCAGGAGCTC TCCATCCCCT |
| 2051 | ACGAGGAGGC GGCGGCCTTC ATCGAGCGCT ACTTCCAGAG CTTCCCCAAG |
| 2101 | GTGCGGGCCT GGATCGCCAA AACCTTGGAG GAGGGGCGGA AGAAGGGGTA |
| 2151 | CGTGGAGACC CTCTTCGGCC GCCGCCGCTA CGTGCCCGAC CTCAACGCCC |
| 2201 | GGGTGAAGAG CGTGCGGGAG GCGGCGGAGC GCATGGCCTT CAACATGCCC |
| 2251 | GTGCAGGGCA CCGCCGCGGA CCTCATGAAG CTGGCCATGG TGAAGCTCTT |
| 2301 | CCCCAGGCTC AGGCCCTTGG GCGTTCGCAT CCTCCTCCAG GTGCACGACG |
| 2351 | AGCTGGTCTT GGAGGCCCCA AAGGCGCGGG CGGAGGAGGC CGCCCAGTTG |
| 2401 | GCCAAGGAGA CCATGGAAGG GGTTTACCCC CTCTCCGTCC CCCTGGAGGT |
| 2451 | GGAGGTGGGG ATGGGGGAGG ACTGGCTTTC CGCCAAGGCC TAG |

The substitution of TAC (encoding Tyr) for TTC (encoding Phe) at the indicated position can reduce discrimination against ddNTP incorporation by DNA polymerase I. See, e.g., U.S. Pat. No. 5,614,365, which is incorporated herein by reference. The substitution of GAC (encoding Asp) for GGG (encoding Gly) at the indicated positions reduces the 5′-3′ exonuclease activity.

The nucleic acids of the invention have homology to portions of the nucleic acids that encode DNA polymerases from Thermus aquaticus and Thermus thermophilus. However, significant portions of the nucleic acid sequences of the present invention are distinct.

The invention also encompasses fragment and variant nucleic acids of SEQ ID NO: 1-4. Nucleic acid “fragments” encompassed by the invention are of two general types. First, fragment nucleic acids that do not encode a full length DNA polymerase but do encode a thermally stable polypeptide with DNA polymerase activity are encompassed within the invention. Second, fragment nucleic acids useful as hybridization probes but that generally do not encode polymerases retaining biological activity are also encompassed within the invention. Thus, fragments of nucleotide sequences such as SEQ ID NO:1-4 may be as small as about 9 nucleotides, about 12 nucleotides, about 15 nucleotides, about 17 nucleotides, about 18 nucleotides, about 20 nucleotides, about 50 nucleotides, about 100 nucleotides or more. In general, a fragment nucleic acid of the invention can have any upper size limit so long as it is related in sequence to the nucleic acids of the invention but is not full length.

As indicated above, “variants” are substantially similar or substantially homologous sequences. For nucleotide sequences, variants include those sequences that, because of the degeneracy of the genetic code, encode the identical amino acid sequence of the native DNA polymerase I protein. Variant nucleic acids also include those that encode polypeptides that do not have amino acid sequences identical to that of a native DNA polymerase I protein, but that encode an active, thermally stable DNA polymerase I with conservative changes in the amino acid sequence.

As is known by one of skill in the art, the genetic code is “degenerate,” meaning that several trinucleotide codons can encode the same amino acid. This degeneracy is apparent from Table 1. Hence, many changes in the nucleotide sequence of the variant may be silent and may not alter the amino acid sequence encoded by the nucleic acid. Where nucleic acid sequence alterations are silent, a variant nucleic acid will encode a polypeptide with the same amino acid sequence as the reference nucleic acid. Therefore, a particular nucleic acid sequence of the invention also encompasses variants with degenerate codon substitutions, and complementary sequences thereof, as well as the sequence explicitly specified by a SEQ ID NO. Specifically, degenerate codon substitutions may be achieved by generating sequences in which the reference codon is replaced by any of the codons for the amino acid specified by the reference codon.

TABLE 1

| 1^{st} | | | | | |
|---|---|---|---|---|---|
| Posi- | Second Position | | | | 3^{rd} |
| tion | T | C | A | G | Position |
| T | TTT = Phe | TCT = Ser | TAT = Tyr | TGT = Cys | T |
| T | TTC = Phe | TCC = Ser | TAC = Tyr | TGC = Cys | C |
| T | TTA = Leu | TCA = Ser | TAA = Stop | TGA = Stop | A |
| T | TTG = Leu | TCG = Ser | TAG = Stop | TGG = Trp | G |
| C | CTT = Leu | CCT = Pro | CAT = His | CGT = Arg | T |
| C | CTC = Leu | CCC = Pro | CAC = His | CGC = Arg | C |
| C | CTA = Leu | CCA = Pro | CAA = Gln | CGA = Arg | A |
| C | CTG = Leu | CCG = Pro | CAG = Gln | CGG = Arg | G |
| A | ATT = Ile | ACT = Thr | AAT = Asn | AGT = Ser | T |
| A | ATC = Ile | ACC = Thr | AAC = Asn | AGC = Ser | C |
| A | ATA = Ile | ACA = Thr | AAA = Lys | AGA = Arg | A |
| A | ATG = Met | ACG = Thr | AAG = Lys | AGG = Arg | G |
| G | GTT = Val | GCT = Ala | GAT = Asp | GGT = Gly | T |
| G | GTC = Val | GCC = Ala | GAC = Asp | GGC = Gly | C |
| G | GTA = Val | GCA = Ala | GAA = Gln | GGA = Gly | A |
| G | GTG = Val | GCG = Ala | GAG = Gln | GGG = Gly | G |

In general, the third position of one or more selected codons can be substituted with mixed-base and/or deoxyinosine residues as disclosed by Batzer et al., Nucleic Acid Res., 19, 5081 (1991) and/or Ohtsuka et al., J. Biol. Chem., 260, 2605 (1985); Rossolini et al., Mol. Cell. Probes, 8, 91 (1994).

However, the invention is not limited to silent changes in the present nucleotide sequences but also includes variant nucleic acid sequences that conservatively alter the amino acid sequence of a polypeptide of the invention. According to the present invention, variant and reference nucleic acids of the invention may differ in the encoded amino acid sequence by one or more substitutions, additions, insertions, deletions, fusions and truncations, which may be present in any combination, so long as an active, thermally stable nucleic acid polymerase is encoded by the variant nucleic acid. Such variant nucleic acids will not encode exactly the same amino acid sequence as the reference nucleic acid, but have conservative sequence changes.

Variant nucleic acids with silent and conservative changes can be defined and characterized by the degree of homology to the reference nucleic acid. Preferred variant nucleic acids are “substantially homologous” to the reference nucleic acids of the invention. As recognized by one of skill in the art, such substantially similar nucleic acids can hybridize under stringent conditions with the reference nucleic acids identified by SEQ ID NOs herein. These types of substantially homologous nucleic acids are encompassed by this invention.

Generally, nucleic acid derivatives and variants of the invention will have at least 90%, 91%, 92%, 93% or 94% sequence identity to the reference nucleotide sequence defined herein. Preferably, nucleic acids of the invention will have at least at least 95%, 96%, 97%, 98%, or 99% sequence identity to the reference nucleotide sequence defined herein.

Variant nucleic acids can be detected and isolated by standard hybridization procedures. Hybridization to detect or isolate such sequences is generally carried out under stringent conditions. “Stringent hybridization conditions” and “stringent hybridization wash conditions” in the context of nucleic acid hybridization experiments such as Southern and Northern hybridization are sequence dependent, and are different under different environmental parameters. Longer sequences hybridize specifically at higher temperatures. An extensive guide to the hybridization of nucleic acids is found in Tijssen, Laboratory Techniques in Biochemistry and Molecular biology-Hybridization with Nucleic Acid Probes, page 1, chapter 2 “Overview of principles of hybridization and the strategy of nucleic acid probe assays” Elsevier, N.Y. (1993). See also, J. Sambrook et al., Molecular Cloning: A Laboratory Manual, Cold Spring Harbor Press, N.Y., pp 9.31-9.58 (1989); J. Sambrook et al., Molecular Cloning: A Laboratory Manual, Cold Spring Harbor Press, N.Y.(3rd ed. 2001).

The invention also provides methods for detection and isolation of derivative or variant nucleic acids encoding nucleic acid polymerase activity. The methods involve hybridizing at least a portion of a nucleic acid comprising SEQ ID NO:1, 2, 3 or 4 to a sample nucleic acid, thereby forming a hybridization complex; and detecting the hybridization complex. The presence of the complex correlates with the presence of a derivative or variant nucleic acid encoding at least a segment of a nucleic acid polymerase. In general, the portion of a nucleic acid comprising SEQ ID NO:1, 2, 3 or 4 used for hybridization is at least fifteen nucleotides, and hybridization is under hybridization conditions that are sufficiently stringent to permit detection and isolation of substantially homologous nucleic acids. In an alternative embodiment, a nucleic acid sample is amplified by the polymerase chain reaction using primer oligonucleotides selected from SEQ ID NO:1, 2, 3 or 4.

Generally, highly stringent hybridization and wash conditions are selected to be about 5° C. lower than the thermal melting point (Tₘ) for the specific double-stranded sequence at a defined ionic strength and pH. For example, under “highly stringent conditions” or “highly stringent hybridization conditions” a nucleic acid will hybridize to its complement to a detectably greater degree than to other sequences (e.g., at least 2-fold over background). By controlling the stringency of the hybridization and/or washing conditions, nucleic acids that are 100% complementary can be identified.

Alternatively, stringency conditions can be adjusted to allow some mismatching in sequences so that lower degrees of similarity are detected (heterologous probing). Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl and 0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The degree of complementarity or homology of hybrids obtained during hybridization is typically a function of post-hybridization washes, the critical factors being the ionic strength and temperature of the final wash solution. The type and length of hybridizing nucleic acids also affects whether hybridization will occur and whether any hybrids formed will be stable under a given set of hybridization and wash conditions. For DNA-DNA hybrids, the Tₘ can be approximated from the equation of Meinkoth and Wahl Anal. Biochem. 138:267-284 (1984); Tₘ 81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Very stringent conditions are selected to be equal to the Tₘ for a particular probe.

An example of stringent hybridization conditions for hybridization of complementary nucleic acids that have more than 100 complementary residues on a filter in a Southern or Northern blot is 50% formamide with 1 mg of heparin at 42° C., with the hybridization being carried out overnight. An example of highly stringent conditions is 0.1 5 M NaCl at 72° C. for about 15 minutes. An example of stringent wash conditions is a 0.2×SSC wash at 65° C. for 15 minutes (see also, Sambrook, infra). Often, a high stringency wash is preceded by a low stringency wash to remove background probe signal. An example of medium stringency for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C.

Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to detect and isolate homologous nucleic acids that are substantially identical to reference nucleic acids of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

In general, Tₘ is reduced by about 1° C. for each 1% of mismatching. Thus, Tₘ, hybridization, and/or wash conditions can be adjusted to hybridize to sequences of the desired sequence identity. For example, if sequences with greater than 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ).

If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). Using these references and the teachings herein on the relationship between Tₘ, mismatch, and hybridization and wash conditions, those of ordinary skill can generate variants of the present nucleic acids.

Computer analyses can also be utilized for comparison of sequences to determine sequence identity. Such analyses include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. Gene 73:237 244 (1988); Higgins et al. CABIOS 5:151-153 (1989); Corpet et al. Nucleic Acids Res. 16:10881-90 (1988); Huang et al. CABIOS 8:155-65 (1992); and Pearson et al. Meth. Mol. Biol. 24:307-331 (1994). The ALIGN program is based on the algorithm of Myers and Miller, supra. The BLAST programs of Altschul et al., J. Mol. Biol. 215:403 (1990), are based on the algorithm of Karlin and Altschul supra. To obtain gapped alignments for comparison purposes, Gapped BLAST (in BLAST 2.0) can be utilized as described in Altschul et al. Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA, 89, 10915 (1989)). See http://www.ncbi.nlm.nih.gov. Alignment may also be performed manually by inspection.

For purposes of the present invention, comparison of nucleotide sequences for determination of percent sequence identity to the polymerase sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Expression of Nucleic Acids Encoding Polymerases

Nucleic acids of the invention may be used for the recombinant expression of the nucleic acid polymerase polypeptides of the invention. Generally, recombinant expression of a polymerase polypeptide of the invention is affected by introducing a nucleic acid encoding that polypeptide into an expression vector adapted for use in particular type of host cell. The nucleic acids of the invention can be introduced and expressed in any host organism, for example, in both prokaryotic or eukaryotic host cells. Examples of host cells include bacterial cells, yeast cells, cultured insect cell lines, and cultured mammalian cells lines. Preferably, the recombinant host cell system is selected that processes and post-translationally modifies nascent polypeptides in a manner similar to that of the organism from which the nucleic acid polymerase was derived. For purposes of expressing and isolating nucleic acid polymerase enzyme polypeptides of the invention, prokaryotic organisms are preferred, for example, Escherichia coli. Accordingly, the invention provides host cells comprising the expression vectors of the invention.

The nucleic acids to be introduced can be conveniently placed in expression cassettes for expression in an organism of interest. Such expression cassettes will comprise a transcriptional initiation region linked to a nucleic acid of the invention. Expression cassettes preferably also have a plurality of restriction sites for insertion of the nucleic acid to be under the transcriptional regulation of various control elements. The expression cassette additionally may contain selectable marker genes. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Preferably the nucleic acid in the vector is under the control of, and operably linked to, an appropriate promoter or other regulatory elements for transcription in a host cell. The vector may be a bi-functional expression vector that functions in multiple hosts. The transcriptional cassette generally includes in the 5′-3′ direction of transcription, a promoter, a transcriptional and translational initiation region, a DNA sequence of interest, and a transcriptional and translational termination region functional in the organism. The termination region may be native with the transcriptional initiation region, may be native with the DNA sequence of interest, or may be derived from another source.

Efficient expression of recombinant DNA sequences in prokaryotic and eukaryotic cells generally requires regulatory control elements directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence that directs both the termination and polyadenylation of the nascent RNA transcript. Efficient polyadenylation of the recombinant transcript is desirable as transcripts lacking a poly A tail are unstable and are rapidly degraded.

Nucleic acids encoding a nucleic acid polymerase may be introduced into bacterial host cells by a method known to one of skill in the art. For example, nucleic acids encoding a thermophilic polymerase of the invention can be introduced into bacterial cells by commonly used transformation procedures such as by treatment with calcium chloride or by electroporation. If the thermophilic polymerase is to be expressed in eukaryotic host cells, nucleic acids encoding the thermophilic polymerase may be introduced into eukaryotic host cells by a number of means including calcium phosphate co-precipitation, spheroplast fusion, electroporation and the like. When the eukaryotic host cell is a yeast cell, transformation may be affected by treatment of the host cells with lithium acetate or by electroporation.

Thus, one aspect of the invention is to provide expression vectors and host cells comprising a nucleic acid encoding a nucleic acid polymerase polypeptide of the invention. A wide range of expression vectors are well known in the art. Description of various expression vectors and how to use them can be found among other places in U.S. Pat. Nos. 5,604,118; 5,583,023; 5,432,082; 5,266,490; 5,063,158; 4,966,841; 4,806,472; 4,801,537; and Goedel et al., Gene Expression Technology, Methods of Enzymology, Vol. 185, Academic Press, San Diego (1989). The expression of polymerases in recombinant cell systems is a well-established technique. Examples of the recombinant expression of DNA polymerase can be found in U.S. Pat. Nos. 5,602,756; 5,545,552; 5,541,311; 5,500,363; 5,489,523; 5,455,170; 5,352,778; 5,322,785; and 4,935,361.

Recombinant DNA and molecular cloning techniques that can be used to help make and use aspects of the invention are described by Sambrook et al., Molecular Cloning: A Laboratory Manual Vol.1-3, Cold Spring Harbor laboratory, Cold Spring Harbor, N.Y. (2001); Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989); and by T. J. Silhavy, M. L. Berman, and L. W. Enquist, Experiments with Gene Fusions, Cold Spring Harbor Laboratory, Cold Spring Harbor, N.Y. (1984).

Nucleic Acid Polymerase Enzymes

The invention provides Thermus oshimai nucleic acid polymerase enzymes, as well as fragments thereof and variant nucleic acid polymerase enzymes that are active and thermally stable. Any polypeptide containing amino acid sequence SEQ ID NO:5, SEQ ID NO:6, SEQ ID NO:7 and SEQ ID NO:8, which are the amino acid sequences for wild type Thermus oshimai and derivative polymerases, are contemplated by the present invention. The polypeptides of the invention are isolated or substantially purified polypeptides. In particular, the isolated polypeptides of the invention are substantially free of proteins normally present in Thermus oshimai bacteria.

In one embodiment, the invention provides a polypeptide of SEQ ID NO:5, which is a wild type Thermus oshimai nucleic acid polymerase polypeptide:

| | | |
|---|---|---|
| 1 | MLPLFEPKGR VLLVDGHHLA YRTFFALKGL TTSRGEPVQA VYGFAKSLLK | 50 |
| 51 | ALKEDGEVAI VVFDAKAPSF RHEAYEAYKA GRAPTPEDFP RQLALIKELV | 100 |
| 101 | DLLGLVRLEV PGFEADDVLA TLAKKAEREG YEVRILSADR DLYQLLSDRI | 150 |
| 151 | HLLHPEGEVL TPGWLQERYG LSPERWVEYR ALVGDPSDNL PGVPGTGEKT | 200 |
| 201 | ALKLLKEWGS LEAILKNLDQ VKPERVREAI RNNLDKLQMS LELSRLRTDL | 250 |
| 251 | PLEVDFAKRR EPDWEGLKAF LERLEFGSLL HEFGLLEAPK EAEEAPWPPP | 300 |
| 301 | GGAFLGFLLS RPEPMWAELL ALAGAKEGRV HRAEDPVGAL KDLKETRGLL | 350 |
| 351 | AKDLSVLALR EGREIPPGDD PMLLAYLLDP GNTNPEGVAR RYGGEWKEDA | 400 |
| 401 | AAPALLSERL WQALYPRVAE EERLLWLYRE VERPLAQVLA HMEATGVRLD | 450 |
| 451 | VPYLEALSQE VAFELERLEA EVHRLAGHPF NLNSRDQLER VLFDELGLPP | 500 |
| 501 | IGKTEKTGKR STSAAVLELL REAHPIVGRI LEYRELMKLK STYIDPLPRL | 550 |
| 551 | VHPKTGRLHT RFNQTATATG RLSSSDPNLQ NIPVRTPLGQ RIRKAFTAEE | 600 |
| 601 | GHLLVALDYS QTELRVLAHL SGDENLIRVF REGKDIHTET AAWMFGVPPE | 650 |
| 651 | GVDGANRRAA KTVNFGVLYG MSAHRLSQEL SIPYEEAAAF IERYFQSFPK | 700 |
| 701 | VRAWTAKTLE EGRKKGYVET LFGRRRYVPD LNARVKSVRE AAERMAFNNP | 750 |
| 751 | VQGTAADLMK LAMVKLFPRL RPLGVRILLQ VHDELVLEAP KARAEEAAQL | 800 |
| 801 | AKETMEGVYP LSVPLEVEVG MGEDWLSAKA 830 | |

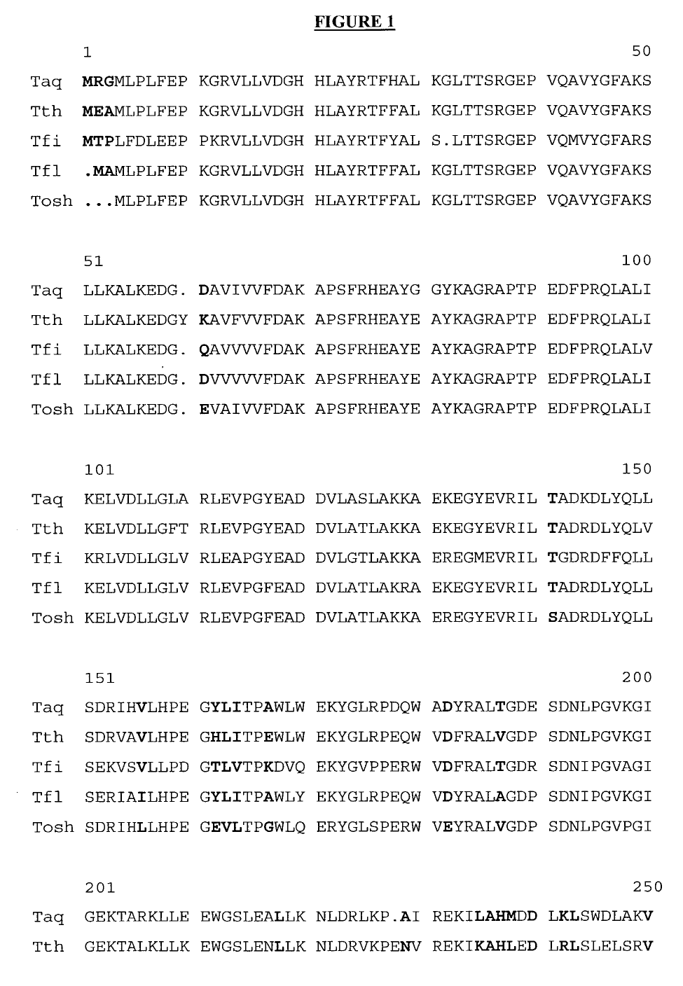

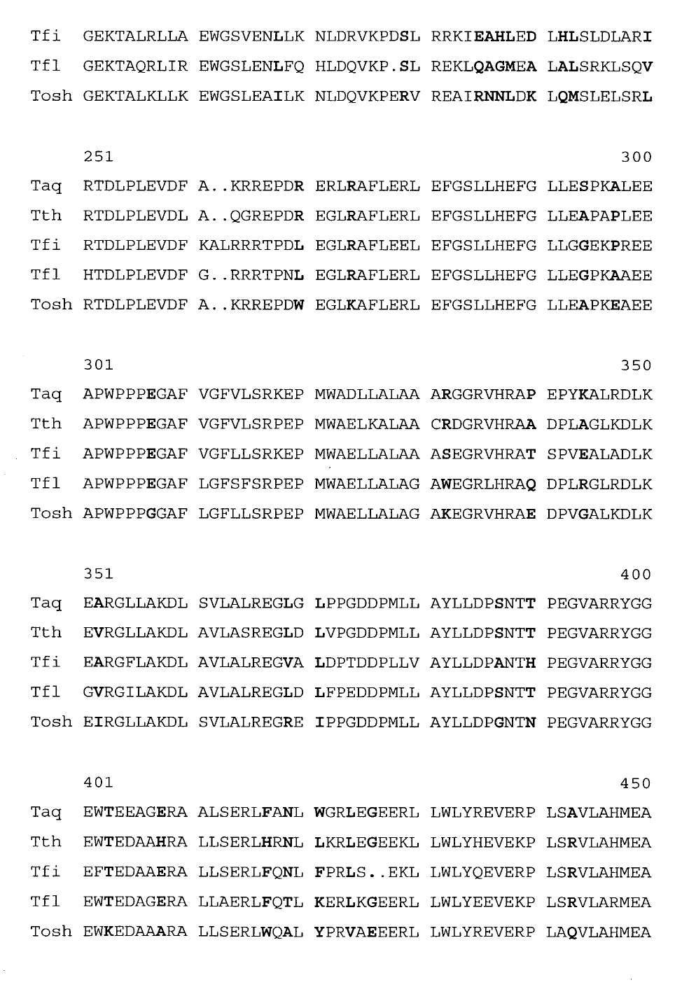

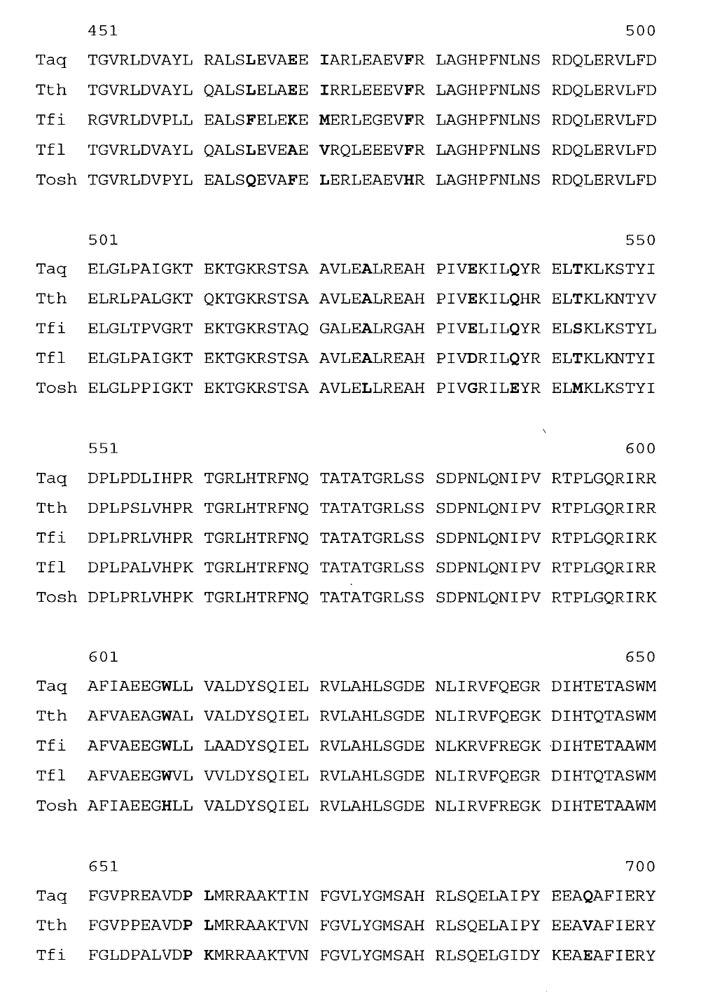

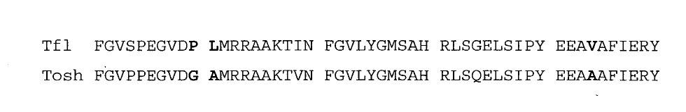

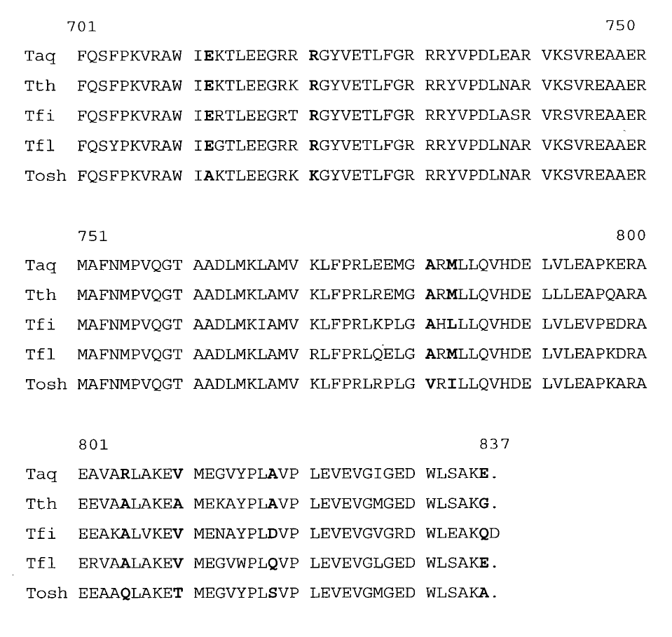

The nucleic acid polymerase polypeptides of the invention have homology to portions of the amino acid sequences of the thermostable DNA polymerases of Thermus aquaticus and Thermus thermophilus (see FIG. 1). However, significant portions of the amino acid sequences of the present invention are distinct, including, for example, amino acids within positions 56-61 (peptide GEVA, SEQ ID NO:9), positions 152-163 (peptide LLHPEGEVLTPG, SEQ ID NO:10), positions 214-256 (peptide ILKNLDQVKPERVREAIRN NLDKLQMSLELSRL, SEQ ID NO:11), positions 264-268 (peptide WEGLK, SEQ ID NO:12), positions 290-293 (peptide KEAE, SEQ ID NO:13), positions 301-305 (peptide GGAFL, SEQ ID NO:14), positions 324-338 (peptide GAKEGRVHRAEDPVG, SEQ ID NO:15), positions 362-365 (peptide GREI, SEQ ID NO:16), positions 381-384 (peptide GNTN, SEQ ID NO:17), positions 396-402 (peptide WKEDAAA, SEQ ID NO:18), positions 411-420 (peptide WQALYPRVAE, SEQ ID NO:19), positions 435-438 (peptide LAQV, SEQ ID NO:20), positions 459-473 (peptide QEVAFE LERLEAEVH, SEQ ID NO:21), positions 519-537 (peptide LLREAHPIVGRILEYRELM, SEQ ID NO:22), positions 601-604 (peptide GHLL, SEQ ID NO:23), positions 651-655 (peptide GVDGA, SEQ ID NO:24), positions 681-689 (peptide SIPYEEAA, SEQ ID NO:25), positions 704-707 (peptide WIAK, SEQ ID NO:26), positions 774-777 (peptide GVRI, SEQ ID NO:27) and positions 799-830 (peptide QLAKETMEGVYPLSVPLEVEVGMGEDWLSAKA, SEQ ID NO:28).

Many polymerases possess activities in addition to a DNA polymerase activity. Such activities include, for example, a 5′-3′ exonuclease activity and/or a 3′-5′ exonuclease activity. The 3′-5′ exonuclease activity improves the accuracy of the newly-synthesized strand by removing incorrect bases that may have been incorporated. DNA polymerases in which such activity is low or absent are prone to errors in the incorporation of nucleotide residues into the primer extension strand. Taq DNA polymerase has been reported to have low 3′-5′ exonuclease activity. See Lawyer et al., J. Biol Chem. 264:6427-6437. In applications such as nucleic acid amplification procedures in which the replication of DNA is often geometric in relation to the number of primer extension cycles, such errors can lead to serious artifactual problems such as sequence heterogeneity of the nucleic acid amplification product (amplicon). Thus, a 3′-5′ exonuclease activity is a desired characteristic of a thermostable DNA polymerase used for such purposes.

By contrast, the 5′-3′ exonuclease activity of polymerase enzymes is often undesirable because this activity may digest nucleic acids, including primers, which have an unprotected 5′ end. Thus, a thermostable polymerase with an attenuated 5′-3′ exonuclease activity, or one in which such activity is absent, is desirable for biochemical applications. Various DNA polymerase enzymes have been described where a modification has been introduced in a DNA polymerase that accomplishes this object. For example, the Klenow fragment of E. coli DNA polymerase I can be produced as a proteolytic fragment of the holoenzyme in which the domain of the protein controlling the 5′-3′ exonuclease activity has been removed. The Klenow fragment still retains the polymerase activity and the 3′-5′ exonuclease activity. Barnes, PCT Publication No. WO92/06188 (1992) and Gelfand et al., U.S. Pat. No. 5,466,591 have produced 5′-3′ exonuclease-deficient recombinant Thermus aquaticus DNA polymerases. Ishino et al., EPO Publication No. 0517418A2, have produced a 5′-3′ exonuclease-deficient DNA polymerase derived from Bacillus caldotenax.

In another embodiment, the invention provides a polypeptide that is a derivative Thermus oshimai polypeptide with reduced or eliminated 5′-3′ exonuclease activity. DNA polymerases that are deficient in 3′-5′ exonuclease activity have superior properties for PCR and for chain termination polynucleotide sequencing. Mutations that reduce 3′-5′ exonuclease activity in DNA polymerase are available to person of ordinary skill in the art. Guidance on how to introduce mutations that reduce 3′-5′ exonuclease activity can be found, among other places, in U.S. Pat. No. 5,466,591; U.S. Pat. No. 5,541,099; U.S. Pat. No. 5,489,523; Bernad et al., Cell 59:219-288(1989); and Xu et al., Biochemical and mutational studies of the 5′-3′ exonuclease of DNA polymerase I of Escherichia coli, J. Mol. Biol. 1997 May 2; 268(2):284-302. In one embodiment, Asp is used in place of Gly at position 43 to produce a polypeptide with reduced 5′-3′ exonuclease activity, and having SEQ ID NO:6, below.

| | | |
|---|---|---|
| 1 | MLPLFEPKGR VLLVDGHHLA YRTFFALKGL TTSRGEPVQA VYDFAKSLLK | 50 |
| 51 | ALKEDGEVAT VVFDAKAPSF RHEAYEAYKA GPAPTPEDFP RQLALIKELV | 100 |
| 101 | DLLGLVRLEV PGFEADDVLA TLAKKAEREG YEVRILSADR DLYQLLSDRT | 150 |
| 151 | HLLHPEGEVL TPGWLQERYG LSPERWVEYR ALVGDPSDNL PGVPGIGEKT | 200 |
| 201 | ALKLLKEWGS LEAILKNLDQ VKPERVREAI RNNLDKLQMS LELSRLRTDL | 250 |
| 251 | PLEVDFAKRR EPDWEGLKAF LERLEFGSLL HEFGLLEAPK EAEEAPWPPP | 300 |
| 301 | GGAFLGFLLS RPEPMWAELL AIAGAKEGRV HRAEDPVGAL KDLKEIRGLL | 350 |
| 351 | AKDLSVLALR EGREIPPGDD PMLLAYLLDP GNTNPEGVAR RYGGEWKEDA | 400 |
| 401 | AARALLSERL WQALYPRVAE EERLLWLYRE VERPLAQVLA HMEATGVRLD | 450 |
| 451 | VPYLEALSQE VAFELERLEA EVHRLAGHPF NLNSRDQLER VLFDELGLPP | 500 |
| 501 | IGKTEKTGKR STSAAVLELL REAHPIVGRI LEYRELMKLK STYIDPLPRL | 550 |
| 551 | VHPKTGRLHT RFNQTATATG RLSSSDPNLQ NIPVRTPLGQ RIRKAFTAEE | 600 |
| 601 | GHLLVALDYS QTELRVLAHL SGDENLTRVF REGKDIHTET AAWMFGVPPE | 650 |
| 651 | GVDGAMRRAA KTVNFGVLYG MSAHRLSQEL SIPYEEAAAF IERYFQSFPK | 700 |
| 701 | VPAWTAKTLE EGRKKGYVET LFGRRRYVPD LNARVKSVRE AAERMAFNMP | 750 |
| 751 | VQGTAADLMK LAMVKLFPRL RPLGVRILLQ VHDELVLEAP KAPAEEAAQL | 800 |
| 801 | AKETMEGVYP LSVPLEVEVG MGEDWLSAKA 830 | |

In another embodiment, the invention provides a polypeptide of SEQ ID NO:7, a derivative Thermus oshimai polypeptide with reduced bias against ddNTP incorporation. SEQ ID NO:7 has Tyr in place of Phe at position 665. The sequence of SEQ ID NO:7 is below.

| | |
|---|---|
| 1 | MLPLFEPKGR VLLVDGHHLA YRTFFALKGL TTSRGEPVQA VYGFAKSLLK 50 |
| 51 | ALKEDGEVAT VVFDAKAPSF RHEAYEAYKA GRAPTPEDFP RQLALIKELV 100 |
| 101 | DLLGLVRLEV PGFEADDVLA TLAKKAEREG YEVRILSADR DLYQLLSDRI 150 |
| 151 | HLLHPEGEVL TPGWLQERYG LSPERWVEYR ALVGDPSDNL PGVPGIGEKT 200 |
| 201 | ALKLLKEWGS LEAILKNLDQ VKPERVREAI RNNLDKLQMS LELSRLRTDL 250 |
| 251 | PLEVDFAKRR EPDWEGLKAF LERLEFGSLL HEFGLLEAPK EAEEAPWPPP 300 |
| 301 | GGAFLGFLLS RPEPMWAELL ALAGAKEGRV HRAEDPVGAL KDLKEIRGLL 350 |
| 351 | AKDLSVLALR EGREIPPGDD PMLLAYLLDP GNTNPEGVAR RYGGEWKEDA 400 |
| 401 | AAPALLSERL WQALYPRVAE EERLLWLYRE VERPLAQVLA HMEATGVRLD 450 |
| 451 | VPYLEALSQE VAFELERLEA EVHRLAGHPF NLNSRDQLER VLFDELGLPP 500 |
| 501 | IGKTEKTGKR STSAAVLELL REAHPTVGRI LEYRELMKLK STYIDPLPRL 550 |
| 551 | VHPKTGRLHT RFNQTATATG RLSSSDPNLQ NIPVRTPLGQ RIRKAFIAEE 600 |
| 601 | GHLLVALDYS QIELRVLAHL SGDENLIRVF REGKDIHTET AAWMFGVPPE 650 |
| 651 | GVDGAMRRAA KTVNYGVLYG MSAHRLSQEL SIPYEEAAAF TERYFQSFPK 700 |
| 701 | VPAWIAKTLE EGRKKGYVET LFGRRRYVPD LNARVKSVRE AAERMAFNMP 750 |
| 751 | VQGTAADLMK TAMVKLFPRL RPLGVRTLLQ VHDELVLEAP KARAEEAAQL 800 |
| 801 | AKETMEGVYP LSVPLEVEVG MGEDWLSAKA 830 |

In another embodiment, the invention provides a polypeptide of SEQ ID NO:8, a derivative Thermus oshimai polypeptide with reduced 5′-3′ exonuclease activity and reduced bias against ddNTP incorporation. SEQ ID NO:8 has Asp in place of Gly at position 43 and Tyr in place of Phe at position 665. The sequence of SEQ ID NO:8 is below.

| | | |
|---|---|---|
| 1 | MLPLFEPKGR VLLVDGHHLA YRTFFALKGL TTSRGEPVQA VYDFAKSLLK | 50 |
| 51 | ALKEDGEVAI VVFDAKAPSF RHEAYEAYKA GRAPTPEDFP RQLALTKELV | 100 |
| 101 | DLLGLVRLEV PGFEADDVLA TLAKKAEREG YEVRTLSADR DLYQLLSDRI | 150 |
| 151 | HLLHPEGEVL TPGWLQERYG LSPERWVEYR ALVGDPSDNL PGVPGTGEKT | 200 |
| 201 | ALKLLKEWGS LEAILKNLDQ VKPERVREAI RNNLDKLQMS LELSRLRTDL | 250 |
| 251 | PLEVDFAKRR EPDWEGLKAF LERLEFGSLL HEFGLLEAPK EAEEAPWPPP | 300 |
| 301 | GGAFLGFLLS RPEPMWAELL ALAGAKEGRV HRAEDPVGAL KDLKEIRGLL | 350 |
| 351 | AKDLSVLALR EGRETPPGDD PMLLAYLLDP GNTNPEGVAR RYGGEWKEDA | 400 |
| 401 | AAPALLSERL WQALYPRVAE EERLLWLYRE VERPLAQVLA HMEATGVRLD | 450 |
| 451 | VPYLEALSQE VAFELERLEA EVHRLAGHPF NLNSRDQLER VLFDELGLPP | 500 |
| 501 | IGKTEKTGKR STSAAVLELL REAHPTVGRT LEYRELMKLK STYTDPLPRL | 550 |
| 551 | VHPKTGRLHT RFNQTATATG RLSSSDPNLQ NIPVRTPLGQ RIRKAFIAEE | 600 |
| 601 | GHLLVALDYS QIELRVLAHL SGDENLIRVF REGKDIHTET AAWMFGVPPE | 650 |
| 651 | GVDGANRPAA KTVNYGVLYG MSAHRLSQEL SIPYEEAAAF IERYFQSFPK | 700 |
| 701 | VPAWTAKTLE EGRKKGYVET LFGRRRYVPD LNARVKSVRE AAERMAFNMP | 750 |
| 751 | VQGTAADLMK LAMVKLFPRL RPLGVRILLQ VHDELVLEAP KARAEEAAQL | 800 |
| 801 | AKETMEGVYP LSVPLEVEVG MGEDWLSAKA 830 | |

As indicated above, derivative and variant polypeptides of the invention are derived from a wild type polymerase by deletion or addition of one or more amino acids to the N-terminal and/or C-terminal end of the wild type polypeptide; deletion or addition of one or more amino acids at one or more sites within the wild type polypeptide; or substitution of one or more amino acids at one or more sites within the wild type polypeptide. Thus, the polypeptides of the invention may be altered in various ways including amino acid substitutions, deletions, truncations, and insertions.

Such variant and derivative polypeptides may result, for example, from genetic polymorphism or from human manipulation. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82, 488 (1985); Kunkel et al., Methods in Enzymol., 154, 367 (1987); U.S. Pat. No. 4,873,192; Walker and Gaastra, eds., Techniques in Molecular Biology, MacMillan Publishing Company, New York (1983) and the references cited therein. Guidance as to appropriate amino acid substitutions that do not affect biological activity of the protein of interest may be found in the model of Dayhoff et al., Atlas of Protein Sequence and Structure, Natl. Biomed. Res. Found., Washington, C.D. (1978), herein incorporated by reference.

The derivatives and variants of the isolated polypeptides of the invention have identity with at least about 92% of the amino acid positions of SEQ ID NO:5, SEQ ID NO:6, SEQ ID NO:7, or SEQ ID NO:8 and have nucleic acid polymerase or DNA polymerase activity and/or are thermally stable. In a preferred embodiment, polypeptide derivatives and variants have identity with at least about 93% of the amino acid positions of SEQ ID NO:5, SEQ ID NO:6, SEQ ID NO:7, or SEQ ID NO:8 and have nucleic acid polymerase or DNA polymerase activity and/or are thermally stable. In a more preferred embodiment, polypeptide derivatives and variants have identity with at least about 95% of the amino acid positions of SEQ ID NO:5, SEQ ID NO:6, SEQ ID NO:7, or SEQ ID NO:8 and have nucleic acid polymerase or DNA polymerase activity and/or are thermally stable.

Amino acid residues of the isolated polypeptides and polypeptide derivatives and variants can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 2.

TABLE 2

| | One-Letter | Common |
|---|---|---|
| Amino Acid | Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| 2,3-Diaminopropionic acid | | Dpr |
| α-Aminoisobutyric acid | | Aib |
| N-Methylglycine (sarcosine) | | MeGly |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3-carboxylic | | |
| acid | | |
| β-2-thienylalanine | | Thi |
| Methionine sulfoxide | | MSO |
| Homoarginine | | hArg |
| N-acetyl lysine | | AcLys |
| 2,4-Diamino butyric acid | | Dbu |
| ρ-Aminophenylalanine | | Phe(pNH₂) |
| N-methylvaline | | MeVal |
| Homocysteine | | hCys |
| Homoserine | | hSer |
| ε-Amino hexanoic acid | | Aha |
| δ-Amino valeric acid | | Ava |
| 2,3-Diaminobutyric acid | | Dab |

Polypeptide variants that are encompassed within the scope of the invention can have one or more amino acids substituted with an amino acid of similar chemical and/or physical properties, so long as these variant polypeptides retain nucleic acid polymerase or DNA polymerase activity and/or remain thermally stable. Derivative polypeptides can have one or more amino acids substituted with an amino acids having different chemical and/or physical properties, so long as these variant polypeptides retain nucleic acid polymerase or DNA polymerase activity and/or remain thermally stable.

Amino acids that are substitutable for each other in the present variant polypeptides generally reside within similar classes or subclasses. As known to one of skill in the art, amino acids can be placed into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include glycine, proline and methionine. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids omithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include cysteine. Examples of non-genetically encoded cysteine-like amino acids include homocysteine and penicillamine.

As will be appreciated by those having skill in the art, the above classifications are not absolute. Several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a polypeptide.

Certain commonly encountered amino acids that are not genetically encoded and that can be present, or substituted for an amino acid, in the variant polypeptides of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); c-aminohexanoic acid (Aha); δ-aminovaleric acid (Ava); N-methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall into the categories defined above.

The classifications of the above-described genetically encoded and non-encoded amino acids are summarized in Table 3, below. It is to be understood that Table 3 is for illustrative purposes only and does not purport to be an exhaustive list of amino acid residues that may comprise the variant and derivative polypeptides described herein. Other amino acid residues that are useful for making the variant and derivative polypeptides described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 3

| Classification | Genetically Encoded | Genetically Non-Encoded |
|---|---|---|
| Hydrophobic | F, L, I, V | |
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl Ala |
| Apolar | M, G, P | |
| Aliphatic | A, V, L, I | t-BuA, t-BuG, MeIle, Nle, MeVal, |
| | | Cha, bAla, MeGly, Aib |
| Hydrophilic | S, K | Cit, hCys |
| Acidic | D, E | |
| Basic | H, K, R | Dpr, Orn, hArg, Phe(p-NH₂), |
| | | DBU, A₂ BU |
| Polar | Q, N, S, T, Y | Cit, AcLys, MSO, hSer |
| Cysteine-Like | C | Pen, hCys, β-methyl Cys |

Polypeptides of the invention can have any amino acid substituted by any similarly classified amino acid to create a variant peptide, so long as the peptide variant is thermally stable and/or retains nucleic acid polymerase or DNA polymerase activity.

“Domain shuffling” or construction of “thermostable chimeric nucleic acid polymerases” may be used to provide thermostable nucleic acid polymerases containing novel properties. For example, placement of codons 289-422 from the Thermus oshimai polymerase coding region after codons 1-288 of the Thermus aquaticus DNA polymerase would yield a novel thermostable polymerase containing the 5′-3′ exonuclease domain of Thermus aquaticus DNA polymerase (1-288), the 3′-5′ exonuclease domain of the Thermus oshimai polymerase (289-422), and the DNA polymerase domain of Thermus aquaticus DNA polymerase (423-832). Alternatively, the 5′-3′ exonuclease domain and the 3′-5′ exonuclease domain of Thermus oshimai polymerase may be fused to the DNA polymerase (dNTP binding and primer/template binding domains) portions of Thermus aquaticus DNA polymerase (about codons 423-832). The donors and recipients need not be limited to Thermus aquaticus and Thermus oshimai polymerases. Thermus thermophilus DNA polymerase 3′-5′ exonuclease, 5′-3′ exonuclease and DNA polymerase domains can similarly be exchanged for those in the Thermus oshimai polymerases of the invention.

It has been demonstrated that the exonuclease domain of Thermus aquaticus Polymerase I can be removed from the amino terminus of the protein with out a significant loss of thermostability or polymerase activity (Erlich et al., (1991) Science 252: 1643-1651, Barnes, W. M., (1992) Gene 112:29-35., Lawyer et al., (1989) JBC 264:6427-6437). Other N-terminal deletions similarly have been shown to maintain thermostability and activity (Vainshtein et al., (1996) Protein Science 5:1785-1792 and references therein.) Therefore this invention also includes similarly truncated forms of any of the wild type or variant polymerases provided herein. For example, the invention is also directed to an active truncated variant of any of the polymerases provided by the invention in which the first 330 amino acids are removed.

Moreover, the invention provides SEQ ID NO:39, a truncated form of a polymerase in which the N-terminal 289 amino acids have been removed from the wild type Thermus oshimai polymerase.

| | | |
|---|---|---|
| 290 | K EAEEAPWPPP | 300 |
| 301 | GGAFLGFLLS RPEPMWAELL ALAGAKEGRV HRAEDPVGAL KDLKBTRGLL | 350 |
| 351 | AKDLSVLALR EGREIPPGDD PMLLAYLLDP GNTNPEGVAR RYGGEWKEDA | 400 |
| 401 | AARALLSERL WQALYPRVAE EERLLWLYRE VERPLAQVLA HMEATGVRLD | 450 |
| 451 | VPYLEALSQE VAFELERLEA EVHRLAGHPF NLNSRDQLER VLFDELGLPP | 500 |
| 501 | IGKTEKTGKR STSAAVLELL REAHPIVGRI LEYRELMKLK STYIDPLPRL | 550 |
| 551 | VHPKTGRLHT RFNQTATATG RLSSSDPNLQ NIPVRTPLGQ RIRKAFIAEE | 600 |
| 601 | GHLLVALDYS QIELRVLAHL SGDENLIRVF REGKDIHTET AAWMFGVPPE | 650 |
| 651 | GVDGAMRRAA KTVNFGVLYG MSAHRLSQEL SIPYEEAAAF TERYFQSFPK | 700 |
| 701 | VPAWIAKTLE EGRKKGYVET LFGRRRYVPD LNARVKSVRE AAERMAFNMP | 750 |
| 751 | VQGTAADLMK LAMVKLFPRL RPLGVRILLQ VHDELVLEAP KARAEEAAQL | 800 |
| 801 | AKETMEGVYP LSVPLEVEVG MGEDWLSAKA 830 | |

Thus, the polypeptides of the invention encompass both naturally occurring proteins as well as variations, truncations and modified forms thereof. Such variants will continue to possess the desired activity. The deletions, insertions, and substitutions of the polypeptide sequence encompassed herein are not expected to produce radical changes in the characteristics of the polypeptide. One skilled in the art can readily evaluate the thermal stability and polymerase activity of the polypeptides and variant polypeptides of the invention by routine screening assays.

Kits and compositions containing the present polypeptides are substantially free of cellular material. Such preparations and compositions have less than about 30%, 20%, 10%, 5%, (by dry weight) of contaminating bacterial cellular protein.

The activity of polymerase polypeptides and variant polypeptides can be assessed by any procedure known to one of skill in the art. For example, the DNA synthetic activity of the variant and non-variant polymerase polypeptides of the invention can be tested in standard DNA sequencing or DNA primer extension reaction. One such assay can be performed in a 100 μl (final volume) reaction mixture, containing, for example, 0.1 mM dCTP, dTTP, dGTP, α-³²P-dATP, 0.3 mg/ml activated calf thymus DNA and 0.5 mg/ml BSA in a buffer containing: 50 mM KCl, 1 mM DTT, 10 MM MgCl₂ and 50 mM of a buffering compound such as PIPES, Tris or Triethylamine. A dilution to 0.1 units/μl of each polymerase enzyme is prepared, and 5 μl of such a dilution is added to the reaction mixture, followed by incubation at 60° C. for 10 minutes. Reaction products can be detected by determining the amount of ³²P incorporated into DNA or by observing the products after separation on a polyacrylamide gel.

Uses for Nucleic Acid Polymerases

The thermostable enzyme of this invention may be used for any purpose in which nucleic acid polymerase enzyme activity is necessary or desired. For example, the present nucleic acid polymerase enzymes can be used in one or more of the following procedures: DNA sequencing, DNA amplification, reverse transcription, RNA amplification, DNA synthesis and/or primer extension. The nucleic acid polymerase enzymes of the invention can be used to amplify DNA or RNA templates by polymerase chain reaction (PCR). The nucleic acid polymerase enzymes of the invention can be used to sequence DNA by Sanger sequencing procedures. The nucleic acid polymerase enzymes of the invention can also be used in primer extension reactions. The nucleic acid polymerase enzymes of the invention can be used test for single nucleotide polymorphisms (SNPs) by single nucleotide primer extension using terminator nucleotides. Any such procedures and related procedures, for example, polynucleotide or primer labeling, minisequencing and the like are contemplated for use with the present nucleic acid polymerase enzymes.

Methods of the invention comprise the step of extending a primed polynucleotide template with at least one labeled nucleotide, wherein the extension is catalyzed by a polymerase of the invention. DNA polymerases used for Sanger sequencing can produce fluorescently labeled products that are analyzed on an automated fluorescence-based sequencing apparatus such as an Applied Biosystems 310 or 377 (Applied Biosystems, Foster City, Calif.). Detailed protocols for Sanger sequencing are known to those skilled in the art and may be found, for example in Sambrook et al, Molecular Cloning, A Laboratory Manual, Second Edition, Cold Spring Harbor Press, Cold Spring Harbor, N.Y. (1989).

In one embodiment, the nucleic acid polymerase enzymes of the invention are used for nucleic acid amplification. Any amplification procedure that employs a DNA polymerase or reverse transcriptase can be used, for example, in polymerase chain reaction (PCR) assays, strand displacement amplification and other amplification procedures. Strand displacement amplification can be used as described in Walker et al (1992) Nucl. Acids Res. 20, 1691-1696. The term “polymerase chain reaction” (“PCR”) refers to the method of K. B. Mullis U.S. Pat. Nos. 4,683,195; 4,683,202; and 4,965,188, hereby incorporated by reference, which describe a method for increasing the concentration of a segment of a target sequence in a mixture of genomic DNA, RNA or other nucleic acids without cloning or purification.

The PCR process for amplifying a target sequence consists of introducing a large excess of two oligonucleotide primers to the mixture of nucleic acids containing the desired target sequence, followed by a precise sequence of thermal cycling in the presence of a polymerase. The two primers are complementary to their respective strands of the double stranded target sequence. To do amplification, the mixture is denatured and the primers are annealed to complementary sequences within the target molecule. Following annealing, the primers are extended with a polymerase so as to form a new pair of complementary strands. The steps of denaturation, primer annealing and polymerase extension are termed a “cycle.” There can be numerous cycles, and the amount of amplified nucleic acid produced increases with each cycle. Hence, to obtain a high concentration of an amplified target nucleic acid, many cycles are performed.

The steps involve in PCR nucleic acid amplification method are described in more detail below. For ease of discussion, the nucleic acid to be amplified is described as being double-stranded. However, the process is equally useful for amplifying a single-stranded nucleic acid, such as an mRNA, although the ultimate product is generally double-stranded DNA. In the amplification of a single-stranded nucleic acid, the first step involves the synthesis of a complementary strand using, for example, one of the two amplification primers. The succeeding steps generally proceed as follows:

(a) Each nucleic acid strand is contacted with four different nucleoside triphosphates and one oligonucleotide primer for each nucleic acid strand to be amplified, wherein each primer is selected to be substantially complementary to a portion the nucleic acid strand to be amplified, such that the extension product synthesized from one primer, when it is separated from its complement, can serve as a template for synthesis of the extension product of the other primer. To promote the proper annealing of primer(s) and the nucleic acid strands to be amplified, a temperature that allows hybridization of each primer to a complementary nucleic acid strand is used.

(b) After primer annealing, a polymerase is used for primer extension that incorporates the nucleoside triphosphates into a growing nucleic acid strand that is complementary to the strand hybridized by the primer. In general, this primer extension reaction is performed at a temperature and for a time effective to promote the activity of the enzyme and to synthesize a “full length” complementary nucleic acid strand, that extends into a through a complete second primer binding. However, the temperature is not so high as to separate each extension product from its nucleic acid template strand.

(c) The mixture from step (b) is then heated for a time and at a temperature sufficient to separate the primer extension products from their complementary templates. The temperature chosen is not so high as to irreversibly denature the polymerase present in the mixture.

(d) The mixture from (c) is cooled for a time and at a temperature effective to promote hybridization of a primer to each of the single-stranded molecules produced in step (b).

(e) The mixture from step (d) is maintained at a temperature and for a time sufficient to promote primer extension by polymerase to produce a “full length” extension product. The temperature used is not so high as to separate each extension product from the complementary strand template. Steps (c)-(e) are repeated until the desired level of amplification is obtained.

The amplification method is useful not only for producing large amounts of a specific nucleic acid sequence of known sequence but also for producing nucleic acid sequences that are known to exist but are not completely specified. One need know only the identity of a sufficient number of bases at both ends of the sequence in sufficient detail so that two oligonucleotide primers can be prepared that will hybridize to different strands of the desired sequence at those positions. An extension product is synthesized from one primer. When that extension product is separated from the template the extension product can serve as a template for extension of the other primer. The greater the knowledge about the bases at both ends of the sequence, the greater can be the specificity of the primers for the target nucleic acid sequence.

Thermally stable polymerases are therefore generally used for PCR because they can function at the high temperatures used for melting double stranded target DNA and annealing the primers during each cycle of the PCR reaction. High temperature results in thermodynamic conditions that favor primer hybridization with the target sequences and not hybridization with non-target sequences (H. A. Erlich (ed.), PCR Technology, Stockton Press [1989]).

The thermostable polymerases of the present invention satisfy the requirements for effective use in amplification reactions such as PCR. The present polymerase polypeptides do not become irreversibly denatured (inactivated) when subjected to the required elevated temperatures for the time necessary to melt double-stranded nucleic acids during the amplification process. Irreversible denaturation for purposes herein refers to permanent and complete loss of enzymatic activity. The heating conditions necessary for nucleic acid denaturation will depend, e.g., on the buffer salt concentration and the composition and length of the nucleic acids being denatured, but typically range from about 90° C. to about 105° C. for a time depending mainly on the temperature and the nucleic acid length, typically from a few seconds up to four minutes. Higher temperatures may be required as the buffer salt concentration and/or GC composition of the nucleic acid is increased. The polymerase polypeptides of the invention do not become irreversibly denatured for relatively short exposures to temperatures of about 90° C. to 100° C.

The thermostable polymerase polypeptides of the invention have an optimum temperature at which they function that is higher than about 45° C. Temperatures below 45° C. facilitate hybridization of primer to template, but depending on salt composition and concentration and primer composition and length, hybridization of primer to template can occur at higher temperatures (e.g., 45° C. to 70° C.), which may promote specificity of the primer hybridization reaction. The polymerase polypeptides of the invention exhibit activity over a broad temperature range from about 37° C. to about 90° C.

The present polymerase polypeptides have particular utility for PCR not only because of their thermal stability but also because of their fidelity in replicating the target nucleic acid. With PCR, it is possible to amplify a single copy of a specific target nucleic acid to a level detectable by several different methodologies. However, if the sequence of the target nucleic acid is not replicated with fidelity, then the amplified product can comprise a pool of nucleic acids with diverse sequences. Hence, a polymerase that can accurately replicate the sequence of the target is highly desirable.

Any nucleic acid can act as a “target nucleic acid” for the PCR methods of the invention. The term “target,” when used in reference to the polymerase chain reaction, refers to the region of nucleic acid bounded by the primers used for polymerase chain reaction. In addition to genomic DNA, any cDNA, RNA, oligonucleotide or polynucleotide can be amplified with the appropriate set of primer molecules. In particular, the amplified segments created by the PCR process itself are, themselves, efficient templates for subsequent PCR amplifications. The length of the amplified segment of the desired target sequence is determined by the relative positions of the primers with respect to each other, and therefore, this length is a controllable parameter.

The amplified target nucleic acid can be detected by any method known to one of skill in the art. For example, target nucleic acids are often amplified to such an extent that they form a band visible on a size separation gel. Target nucleic acids can also be detected by hybridization with a labeled probe; by incorporation of biotinylated primers during PCR followed by avidin-enzyme conjugate detection; by incorporation of ³²P-labeled deoxynucleotide triphosphates during PCR, and the like.

The amount of amplification can also be monitored, for example, by use of a reporter-quencher oligonucleotide as described in U.S. Pat. No. 5,723,591, and a DNA polymerase of the invention that has 5′-3′ nuclease activity. The reporter-quencher oligonucleotide has an attached reporter molecule and an attached quencher molecule that is capable of quenching the fluorescence of the reporter molecule when the two are in proximity. Quenching occurs when the reporter-quencher oligonucleotide is not hybridized to a complementary nucleic acid because the reporter molecule and the quencher molecule tend to be in proximity or at an optimal distance for quenching. When hybridized, the reporter-quencher oligonucleotide emits more fluorescence than when unhybridized because the reporter molecule and the quencher molecule tend to be further apart. To monitor amplification, the reporter-quencher oligonucleotide is designed to hybridize 3′ to an amplification primer. During amplification, the 5′-3′ nuclease activity of the polymerase digests the reporter oligonucleotide probe, thereby separating the reporter molecule from the quencher molecule. As the amplification is conducted, the fluorescence of the reporter molecule increases. Accordingly, the amount of amplification performed can be quantified based on the increase of fluorescence observed.

Oligonucleotides used for PCR primers are usually about 9 to about 75 nucleotides, preferably about 17 to about 50 nucleotides in length. Preferably, an oligonucleotide for use in PCR reactions is about 40 or fewer nucleotides in length (e.g., 9, 12, 15, 18, 20, 21, 24, 27, 30, 35, 40, or any number between 9 and 40). Generally specific primers are at least about 14 nucleotides in length. For optimum specificity and cost effectiveness, primers of 16-24 nucleotides in length are generally preferred.

Those skilled in the art can readily design primers for use processes such as PCR. For example, potential primers for DNA amplification can be used as probes to determine whether the primer is selective for a single target and what conditions permit hybridization of a primer to a target within a sample or complex mixture of nucleic acids.

The present invention also contemplates use of the present polymerase polypeptides in combination with other procedures or enzymes. For example, the polymerase polypeptides can be used for reverse transcription of a specific RNA. In this method, the RNA is converted to cDNA due to the reverse transcriptase activity of the present polymerases, and then amplified using the same polymerase. No addition of a separate RNA polymerase may be needed. Procedures for reverse transcription and amplification of RNA are provided in U.S. Pat. No. 5,322,770, incorporated by reference herein.

In another embodiment, polymerases of the invention with 5′-3′ exonuclease activity are used to detect target nucleic acids in an invader-directed cleavage assay. This type of assay is described, for example, in U.S. Pat. 5,994,069. It is important to note that the 5′-3′ exonuclease of DNA polymerases is not really an exonuclease that progressively cleaves nucleotides from the 5′ end of a nucleic acid, but rather a nuclease that can cleave certain types of nucleic acid structures to produce oligonucleotide cleavage products. Such cleavage is sometimes called structure-specific cleavage.

In general, the invader-directed cleavage assay employs at least one pair of oligonucleotides that interact with a target nucleic acid to form a cleavage structure for the 5′-3′ nuclease activity of the polymerase. Distinctive cleavage products are released when the cleavage structure is cleaved by the 5′-3′ nuclease activity of the polymerase. Formation of such a target-dependent cleavage structure and the resulting cleavage products is indicative of the presence of specific target nucleic acid sequences in the test sample.

Therefore, in the invader-directed cleavage procedure, the 5′-3′ nuclease activity of the present polymerases is needed as well at least one pair of oligonucleotides that interact with a target nucleic acid to form a cleavage structure for the 5′-3′ nuclease. The first oligonucleotide, sometimes termed the “probe,” can hybridize within the target site but downstream of a second oligonucleotide, sometimes termed an “invader” oligonucleotide. The invader oligonucleotide can hybridize adjacent and upstream of the probe oligonucleotide. However, the target sites to which the probe and invader oligonucleotides hybridize overlap such that the 3′ segment of the invader oligonucleotide overlaps with the 5′ segment of the probe oligonucleotide. The 5′-3′ nuclease of the present polymerases can cleave the probe oligonucleotide at an internal site to produce distinctive fragments that are diagnostic of the presence of the target nucleic acid in a sample. Further details and methods for adapting the invader-directed cleavage assay to particular situations can be found in U.S. Pat. No. 5,994,069.

One or more nucleotide analogs can also be used with the present methods, kits and with the polymerase polypeptides. Such nucleotide analogs can be modified or non-naturally occurring nucleotides such as 7-deaza purines (i.e., 7-deaza-dATP and 7-deaza-dGTP). Nucleotide analogs include base analogs and comprise modified forms of deoxyribonucleotides as well as ribonucleotides. As used herein the term “nucleotide analog” when used in reference to targets present in a PCR mixture refers to the use of nucleotides other than dATP, dGTP, dCTP and dTTP; thus, the use of dUTP (a naturally occurring dNTP) in a PCR would comprise the use of a nucleotide analog in the PCR. A PCR product generated using dUTP, 7-deaza-dATP, 7-deaza-dGTP or any other nucleotide analog in the reaction mixture is said to contain nucleotide analogs.

The invention also provides kits that contain at least one of the polymerase polypeptides of the invention. Individual kits may be adapted for performing one or more of the following procedures: DNA sequencing, DNA amplification, RNA Amplification and/or primer extension. Kits of the invention comprise a polymerase polypeptide of the invention and at least one nucleotide. A nucleotide provided in the kits of the invention can be labeled or unlabeled. Kits preferably can also contain instructions on how to perform the procedures for which the kits are adapted.

Optionally, the subject kit may further comprise at least one other reagent required for performing the method the kit is adapted to perform. Examples of such additional reagents include: another unlabeled nucleotide, another labeled nucleotide, a balance mixture of nucleotides, one or more chain terminating nucleotides, one or more nucleotide analogs, buffer solution(s), magnesium solution(s), cloning vectors, restriction endonucleases, sequencing primers, reverse transcriptase, and DNA or RNA amplification primers. The reagents included in the kits of the invention may be supplied in premeasured units so as to provide for greater precision and accuracy. Typically, kits reagents and other components are placed and contained in separate vessels. A reaction vessel, test tube, microwell tray, microtiter dish or other container can also be included in the kit. Different labels can be used on different reagents so that each reagent can be distinguished from another.

The following Examples further illustrate the invention and are not intended to limit the scope of the invention.

### EXAMPLE 1

### Cloning of Gene Encoding a Thermus oshimai Nucleic Acid Polymerase

Growth of bacteria and genomic DNA isolation

Thermus oshimai polymerase nucleic acids were cloned from the strain designated CB1. The source for the isolate is the Azores (from Dr. R. A. D. Williams, Queen Mary and Westfield College, London, England). A lyophilized sample of Thermus oshimai strain CB1 was obtained, revived in ATCC Culture Medium 461 (Castenholz TYE medium) and grown overnight to stationary phase. Thermus oshimai genomic DNA was prepared using a Qiagen genomic DNA preparation protocol and kit (Qiagen Inc., Valencia, Calif.).

Cloning methods

The first forward and reverse primers were designed by analysis of 5′ and 3′ terminal homologous conserved regions of the DNA sequences of Thermus aquaticus (Taq), Thermus thermophilus (Tth), Thermus filiformis (Tfi), Thermus caldophilus, and Thermus flavus (Tfl). A partial Thermus oshimai polymerase gene fragment was amplified using N-terminal primer 5′ ggc cac cac ctg gcc tac 3′ (SEQ ID NO:29) and the C-terminal primer 5′ ccc acc tcc acc tcc ag 3′ (SEQ ID NO:30). The PCR reaction mixture contained 2.5 μl of 10×Amplitaq buffer (ABi), 2 mM MgCl, 100 ng DNA template, 2.5 mM (each) dNTP, 20 pmol of each primer, and 1.25 units of Amplitaq DNA polymerase in a 25 μl total reaction volume

The PCR reaction mixture was cycled 30 times (97° C. for 3 sec; 56° C. for 30 sec; 72° C. for 3 min) with a finishing step (72° C. for 7 min). This produced a DNA fragment of approximately 2.4 kb that was purified from the PCR reaction mix using a Qiagen PCR cleanup kit (Qiagen). The Thermus oshimai strain CB1 fragment was ligated into the vector pCR®T7 CT-TOPO® (Invitrogen, Carlsbad, Calif.). The sequence of the cloned fragment showed some homology to the Thermus thermophilus Pol I gene (Genebank accession number 466573) between nucleotides 644 and 3060.

To obtain the sequence of the full-length Thermus oshimai polymerase gene a library was constructed by SmaI digestion of Thermus oshimai genomic DNA. After complete digestion, the resulting fragments were ligated to the GenomeWalker™ adapter (Universal GenomeWalker™ kit, Clonetech, Mountainview, Calif.). A fragment containing the 5′-terminal portion of the Thermus oshimai polymerase gene was amplified using a gene specific primer (5′-catcctccttcagggccttga-3′, SEQ ID NO:3 1) and Clonetech adapter primer API (Universal Genome Walker kit, Clonetech, Mountainview, Calif.). The PCR mixture was heated to 80° and then the gene-specific primer was added. The cycling conditions were 7 cycles (94° C. for 25 sec, 72° C. for 3 min) followed by 30 cycles (96° C. for 5 sec, 66° for 1 min). The amplified fragment from the GenomeWalker™ library was purified from the PCR reaction mix using a Quiagen PCR cleanup kit (Quiagen). The fragment was sequenced using the same gene-specific primer used for amplification. To obtain the sequence of the 3′ end of the Thermus oshimai polymerase gene the above process was repeated with the same Sma I library using the gene-specific primer 5′-ttg cag gtg cac gac gag ctg gtc-3′ (SEQ ID NO:32). A fragment containing the 3′ end of the Thermus oshimai polymerase gene was sequenced with the same gene-specific primer. These sequences were assembled with the sequence of the first gene fragment to obtain the entire Thermus oshimai nucleic acid polymerase gene. The DNA sequence of the N terminal end of the Thermus oshimai gene (start codon in boldface) is 5′-atg ctg ccc ctc ttt gag ccc aag ggc cgg-3′ (SEQ ID NO:33). The C-terminal end of the Thermus oshimai polymerase gene (stop codon in boldface) has the sequence 5′-gtg ggg atg ggg gag gac tgg ctt tcc gcc aag gcc tag-3′ (SEQ ID NO:34).

Two primers were used to PCR amplify a product containing the full-length, wild-type Thermus oshimai polymerase gene (SEQ ID NO:1). The primer complementary to the 3′ terminal sequence was 5′-gtc gac tag gcc ttg gcg aaa gcc a-3′ (SEQ ID NO:35). It has a SalI recognition site (gtcgac, (SEQ ID NO:36) overlapping with the stop codon. The primer complementary to the 5′ terminal was 5′-cat atg ctt ccc ctc ttt gag ccc a-3′ (SEQ ID NO:37). The recognition site for restriction enzyme NdeI (catatg, SEQ ID NO:38) was introduced using this primer. The NdeI and SalI restriction sites were used to facilitate subcloning of the gene into other plasmid DNA vectors. The PCR reaction mixture used to amplify Thermus oshimai polymerase gene from strain CB1 contained 2.5 μl of 10×Amplitaq reaction buffer (Applied Biosystems), 2 mM MgCl₂, 120 ng genomic DNA template, 0.2 mM (each) dNTPs, 20 pmol of each primer, and 1.25 units of Amplitaq in a 25 μl total reaction volume. The reaction was started by adding a premix containing enzyme, MgCl₂, dNTPs, buffer and water to another premix containing primer and template preheated at 80° C. The entire reaction mixture was then denatured (96° C., 30 sec) followed by 30 PCR cycles (97° C. for 3 sec; 62° C. for 30 sec; 72° C. for 3 min) with a finishing step (72° C. for 7 min).

This PCR reaction produced an approximate 2.5 kb DNA fragment. The amplified fragment was purified from the PCR reaction mix using a Qiagen PCR cleanup kit (Qiagen). The Thermus oshimai strain CB1 fragment was ligated into the inducible expression vector pCR®T7 CT-TOPO® (Invitrogen, Carlsbad, Calif.). Three different cloned Thermus oshimai polymerase genes were sequenced independently in order to rule out PCR errors. The resulting consensus sequence is the reported Thermus oshimai nucleic acid polymerase sequence of this invention.

Modification of Wild Type Thermus oshimai Polymerase Gene

In order to produce Thermus oshimai polymerase in a form suitable for dye-terminator DNA sequencing, two amino acid substitutions were made. These are the FS mutation (U.S. Pat. No. 5,614,365; Tabor and Richardson, 1995 PNAS 92: 6339-6343) and exo-minus mutation (U.S. Pat. No. 5,466,591; Xu et al., Biochemical and mutational studies of the 5′-3′ exonuclease of DNA polymerase I of Escherichia coli, J. Mol. Biol. 1997 May 2; 268(2):284-302). The Thermus oshimai G43D variant polypeptide reduces the exonuclease activity to very low levels and Thermus oshimai F665Y variant polypeptide reduces discrimination between ddNTP's and dNTP's. Mutagenesis was carried out using the modified QuickChange™ (Stratagene) PCR mutagenesis protocol described in Sawano & Miyawaki (2000), Nucleic Acids Research Vol. 28. The mutated gene was resequenced completely to confirm the introduction of the mutations and to ensure that no PCR errors were introduced.

### EXAMPLE 2

### Protein Expression and Purification

The cells were grown in one liter of Terrific Broth (Maniatis) to an optical density of 1.2 OD and the protein was overproduced by four-hour induction with 1.0 mM IPTG. The cells were harvested by centrifugation, washed in 50 mM Tris (pH 7.5), 5 mM EDTA, 5% glycerol, 10 mM EDTA to remove growth media, and the cell pellet frozen at −80° C.

To isolate the Thermus oshimai polymerase, the cells were thawed and resuspended in 2.5 volumes (wet weight) of 50 mM Tris (pH 7.2), 400 mM NaCl, 1 mM EDTA. The cell walls were disrupted by sonication and the resulting E. coli cell debris was removed by centrifugation. The resulting lysate was pasteurized in a water bath (75° C. for 45 min), denaturing and precipitating the majority of the non-thermostable E. coli proteins and leaving the thermostable Tosh Pol I in solution. E. coli genomic DNA was removed by coprecipitation with 0.3% Polyethyleneimine (PET). The cleared lysate is then applied to two columns in series: (1) a Biorex 70 cation exchange resin that chelates excess PEI and (2) a heparin-agarose column (dimensions to be provided) that retains the polymerase. This column Case No. 4777 US is washed with 5 column volumes of 20 mM Tris (pH 8.5), 5%glycerol, 100 M NaCl, 0.1 mM EDTA, 0.05% Triton X-100 and 0.05% Tween-20 (KTA). The protein was then eluted with a 0.1 to 1.0 M NaCl linear gradient. The polymerase eluted at 0.8M NaCl. The eluted Thermus oshimai polymerase was concentrated and the buffer exchanged using a Millipore concentration filter (30 kD M.W. cutoff). The concentrated protein was stored at in KTA (no salt) plus 50% glycerol at −20° C.

The activity of the polymerase was measured using the standard salmon sperm DNA radiometric activity assay and sequencing was tested using the Big Dye Version 3. The enzyme is active in 40-80 mM Tris, 1.0-2.0 mM MgCl at a dNTP mix consisting of 0.2 mM dATP, 0.2 mM dCTP, 0.2 mM dUTP, and 0.3 mM dITP, at pH 8.0-10.0, with optimal activity between pH 9.0 and 9.58. The enzyme is also active in KCl concentrations from 0 to 100 mM, indicating that the T. oshimai polymerase is more salt-tolerant than either Tfil or Taq, but not quite as salt-tolerant at Tth.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant arts are intended to be within the scope of the following claims.

What is claimed is:

1. 1. An isolated nucleic acid encoding a nucleic acid polymerase comprising amino acid sequence SEQ ID NO:5, SEQ ID NO:6, SEQ ID NO:7 or SEQ ID NO:8.

2. An isolated nucleic acid encoding a derivative nucleic acid polymerase comprising amino acid sequence SEQ ID NO:5 having a mutation that decreases 5-3′ exonuclease activity.

3. The isolated nucleic acid of claim 2, wherein the derivative nucleic acid polymerase has decreased 5-3′ exonuclease activity relative to a nucleic acid polymerase comprising amino acid sequence SEQ ID NO:5.

4. An isolated nucleic acid encoding a derivative nucleic acid polymerase comprising amino acid sequence SEQ ID NO:5 having a mutation that reduces discrimination against dideoxynucleotide triphosphates.

5. The isolated nucleic acid of claim 5, wherein the derivative nucleic acid polymerase has reduced discrimination against dideoxynucleotide triphosphates relative to a nucleic acid polymerase comprising amino acid sequence SEQ ID NO:5.

6. An isolated nucleic acid encoding a nucleic polymerase comprising SEQ ID NO:1, SEQ ID NO:2, SEQ ID NO:3, or SEQ ID NO:4.

7. An isolated nucleic acid comprising a sequence complementary to SEQ ID NO:1, SEQ ID NO:2, SEQ ID NO:3, or SEQ ID NO:4.

8. A vector comprising an isolated nucleic acid encoding a nucleic acid polymerase comprising amino acid sequence SEQ ID NO:5, SEQ ID NO:6, SEQ ID NO:7 or SEQ ID NO:8.

9. A vector comprising an isolated nucleic acid encoding a derivative nucleic acid polymerase comprising amino acid sequence SEQ ID NO:5 having a mutation that decreases 5-3′ exonuclease activity.

10. The vector of claim 9, wherein the derivative nucleic acid polymerase has decreased 5-3′ exonuclease activity relative to a nucleic acid polymerase comprising amino acid sequence SEQ ID NO:5.

11. A vector comprising an isolated nucleic acid encoding a derivative nucleic acid polymerase comprising amino acid sequence SEQ ID NO:5 having a mutation that reduces discrimination against dideoxynucleotide triphosphates.

12. The vector of claim 11, wherein the derivative nucleic acid polymerase has reduced discrimination against dideoxynucleotide triphosphates relative to a nucleic acid polymerase comprising amino acid sequence SEQ ID NO:5.

13. A vector comprising an isolated nucleic acid encoding a nucleic polymerase comprising SEQ ID NO:1, SEQ ID NO:2, SEQ ID NO:3, or SEQ ID NO:4.

14. An expression vector comprising a promoter operably linked to an isolated nucleic acid encoding a nucleic acid polymerase comprising amino acid sequence SEQ ID NO:5, SEQ ID NO:6, SEQ ID NO:7 or SEQ ID NO:8.

15. An expression vector comprising a promoter operably linked to an isolated nucleic acid encoding a derivative nucleic acid polymerase comprising amino acid sequence SEQ ID NO:5 having a mutation that decreases 5-3′ exonuclease activity.

16. The expression vector of claim 15, wherein the derivative nucleic acid polymerase has decreased 5-3′ exonuclease activity relative to a nucleic acid polymerase comprising amino acid sequence SEQ ID NO:5.

17. An expression vector comprising a promoter operably linked to an isolated nucleic acid encoding a derivative nucleic acid polymerase comprising amino acid sequence SEQ ID NO:5 having a mutation that reduces discrimination against dideoxynucleotide triphosphates.

18. The expression vector of claim 17, wherein the derivative nucleic acid polymerase has reduced discrimination against dideoxynucleotide triphosphates relative to a nucleic acid polymerase comprising amino acid sequence SEQ ID NO:5.

19. An expression vector comprising a promoter operably linked to an isolated nucleic acid encoding a nucleic polymerase comprising SEQ ID NO:1, SEQ ID NO:2, SEQ ID NO:3, or SEQ ID NO:4.

20. The host cell of claim 19, wherein the host cell can express a thermostable polypeptide encoded by the nucleic acid, and the polypeptide has DNA polymerase activity.

21. A host cell comprising an isolated nucleic acid encoding a nucleic acid polymerase comprising amino acid sequence SEQ ID NO:5, SEQ ID NO:6, SEQ ID NO:7 or SEQ ID NO:8.

22. A host cell comprising an isolated nucleic acid encoding a derivative nucleic acid polymerase comprising amino acid sequence SEQ ID NO:5 having a mutation that decreases 5-3′ exonuclease activity.

23. The host cell of claim 22, wherein the derivative nucleic acid polymerase has decreased 5-3′ exonuclease activity relative to a nucleic acid polymerase comprising amino acid sequence SEQ ID NO:5.

24. A host cell comprising an isolated nucleic acid encoding a derivative nucleic acid polymerase comprising amino acid sequence SEQ ID NO:5 having a mutation that reduces discrimination against dideoxynucleotide triphosphates.

25. The host cell of claim 24, wherein the derivative nucleic acid polymerase has reduced discrimination against dideoxynucleotide triphosphates relative to a nucleic acid polymerase comprising amino acid sequence SEQ ID NO:5.

26. A host cell comprising an isolated nucleic acid encoding a nucleic polymerase comprising SEQ ID NO:1, SEQ ID NO:2, SEQ ID NO:3, or SEQ ID NO:4.

27. An isolated nucleic acid polymerase comprising amino acid sequence SEQ ID NO:5, SEQ ID NO:6, SEQ ID NO:7 or SEQ ID NO:8.

28. An isolated derivative nucleic acid polymerase comprising SEQ ID NO:5 having a mutation that decreases 5-3′ exonuclease activity.

29. The isolated derivative nucleic acid polymerase of claim 28, wherein the derivative nucleic acid polymerase has decreased 5-3′ exonuclease activity relative to a nucleic acid polymerase comprising amino acid sequence SEQ ID NO:5.

30. An isolated derivative nucleic acid polymerase comprising SEQ ID NO:5 having a mutation that reduces discrimination against dideoxynucleotide triphosphates.

31. The isolated derivative nucleic acid polymerase of claim 30, wherein the derivative nucleic acid polymerase has reduced discrimination against dideoxynucleotide triphosphates relative to a nucleic acid polymerase comprising amino acid sequence SEQ ID NO:5.

32. A kit comprising a container containing a nucleic acid polymerase, wherein the nucleic acid polymerase comprises an amino acid sequence SEQ ID NO:5, SEQ ID NO:6, SEQ ID NO:7 or SEQ ID NO:8.

33. The kit of claim 32 further comprising a container containing an unlabeled nucleotide, a labeled nucleotide, a balanced mixture of nucleotides, a chain terminating nucleotide, a nucleotide analog, a buffer solution, a solution containing magnesium, a cloning vector, a restriction endonuclease, a sequencing primer, a solution containing reverse transcriptase, or a DNA or RNA amplification primer.

34. The kit of claim 32, adapted for performing DNA sequencing, DNA amplification, reverse transcription, RNA amplification or primer extension reactions.

35. A method of making a nucleic acid polymerase comprising SEQ ID NO:5, SEQ ID NO:6, SEQ ID NO:7 or SEQ ID NO:8 that comprises, incubating under conditions sufficient for RNA transcription and translation a host cell comprising a nucleic acid encoding a polypeptide comprising SEQ ID NO:5, SEQ ID NO:6, SEQ ID NO:7 or SEQ ID NO:8 operably linked to a promoter.

36. The method of claim 35 wherein the nucleic acid comprises SEQ ID NO:1, SEQ ID NO:2, SEQ ID NO:3, or SEQ ID NO:4.

37. A nucleic acid polymerase made by the method of claim 35.

38. A method of synthesizing DNA comprising contacting a polypeptide comprising SEQ ID NO:5, SEQ ID NO:6, SEQ ID NO:7 or SEQ ID NO:8 with a DNA under conditions sufficient to permit polymerization of DNA.

39. A method for thermocyclic amplification of nucleic acid comprising:
(a) contacting a nucleic acid with a thermostable polypeptide having SEQ ID NO:5, SEQ ID NO:6, SEQ ID NO:7 or SEQ ID NO:8 under conditions suitable for amplification of said nucleic acid; and
(b) amplifying the nucleic acid.

40. The method of claim 39 wherein the thermocyclic amplification of the nucleic acid includes cycles of denaturation, primer annealing and primer extension.

41. The method of claim 39 wherein the thermocyclic amplification of the nucleic acid is performed by Strand Displacement Amplification.

42. The method of claim 39 wherein the thermocyclic amplification of DNA is performed by Polymerase Chain Reaction.

43. A method of primer extending DNA comprising contacting a polypeptide comprising SEQ ID NO:5, SEQ ID NO:6, SEQ ID NO:7 or SEQ ID NO:8 with a nucleic acid under conditions sufficient to permit polymerization of DNA.

44. The method of claim 43 wherein the nucleic acid is DNA.

45. The method of claim 43 wherein the primer extension is done to sequence the nucleic acid.

46. The method of claim 43 wherein the primer extension is done to amplify the nucleic acid.
